SEQUENCE LISTING (33 sequences)

SEQ ID NO: 1 - protein, 698 aa, Artificial Sequence
  PCSK9 with a His tag PCSK9-His6, used as an immunogen for immunizing mice or used as a test reagent
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQHHHHHH

SEQ ID NO: 2 - protein, 714 aa, Artificial Sequence
  PCSK9 with a PADRE peptide and a His-tag PCSK9-PADRE-His6, used as an immunogen, wherein the contained PADRE peptide can promote immunization
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQGSGAKFVAAWTLKAAAHHHHHH

SEQ ID NO: 3 - protein, 704 aa, Artificial Sequence
  A fusion protein of PCSK9 with a TEV cleavage site and a His tag PCSK9-TEV-His6, N-PCSK9 (N terminal PCSK9 domain), used as an immunogen, which can be obtained by TEV cleavage
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHENLYFQGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQHHHHHH

SEQ ID NO: 4 - protein, 698 aa, Artificial Sequence
  PCSK9-D374Y mutant protein, with a His-tag PCSK9-D374Y-His6, used as a test reagent
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSYCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQHHHHHH

SEQ ID NO: 5 - protein, 719 aa, Artificial Sequence
  PCSK9 protein inserted with a biotin receiving peptide BP15 and a His tag
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQGSTSGSGLNDIFEAQKIEWHEHHHHHH

SEQ ID NO: 6 - protein, 719 aa, Artificial Sequence
  PCSK9 D374Y mutant protein inserted with a biotin receiving peptide BP15 and a His tag PCSK9-D374Y-BP15- His6, as a test protein
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSYCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQGSTSGSGLNDIFEAQKIEWHEHHHHHH

SEQ ID NO: 7 - protein, 802 aa, Artificial Sequence
  PCSK9 receptor protein LDLR extracellular domain with a Flag tag and a His tag LDLR-ECD-Flag-His6 as a test reagent
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRDYKDDDDKHHHHHH

SEQ ID NO: 8 - protein, 331 aa, Artificial Sequence
  LCDR-Fc, a fusion protein of truncated LDLR extracellular domain with a hIgG1 Fc (with PCSK9 binding activity) LDLR -sECD CFc (hIgG1) as a test reagent
MEFGLSWLFLVAILKGVQCGTNECLDNNGGCSHVCNDLKIGYECLCPDGFQLVAQRRCEDIDECQDPDTCSQLCVNLEGGYKCQCEEGFQLDPHTKACKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 294 aa, Artificial Sequence
  A fusion protein of a more truncated LDLR extracellular domain with a hIgG1 Fc (with PCSK9 binding activity) LDLR-ssECD CFc (hIgG1) as a test reagent
MEFGLSWLFLVAILKGVQCGTNECLDNNGGCSHVCNDLKIGYECLCPDGFQLVAQRRCEDIDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 121 aa, Mus musculus
QVHLQQSGAELAKPGASVKLSCKASGYTFNDYWMHWVKERPGQGLEWIGYINPSSGFTKYHQNFKDKATLTADKSSSTAYMQLSSLTYDDSAVYYCARQYDYDEDWYFDVWGTGTTVTVSS

SEQ ID NO: 11 - protein, 112 aa, Mus musculus
DIVMSQSPSSLAVSAGEKVTMSCKSSQSLLNSRTRKNFLAWYQQKPGQSPKLLIYWASTRESGVPDRFTGRGSGTDFTLTISSVQAEDLAVYYCKQSFNLFTFGSGTKLEIK

SEQ ID NO: 12 - protein, 5 aa, Mus musculus
DYWMH

SEQ ID NO: 13 - protein, 17 aa, Mus musculus
YINPSSGFTKYHQNFKD

SEQ ID NO: 14 - protein, 12 aa, Mus musculus
QYDYDEDWYFDV

SEQ ID NO: 15 - protein, 17 aa, Mus musculus
KSSQSLLNSRTRKNFLA

SEQ ID NO: 16 - protein, 7 aa, Mus musculus
WASTRES

SEQ ID NO: 17 - protein, 8 aa, Mus musculus
KQSFNLFT

SEQ ID NO: 18 - protein, 121 aa, Artificial Sequence
  ch-001
QVQLVQSGAEVKKPGASVKVSCKASGYTFTDYWMHWVRQAPGQGLEWMGYINPSSGFTKYHQNFKDRVTMTRDTSISTAYMELSRLRSDDTAVYYCARQYDYDEDWYFDVWGQGTTVTVSS

SEQ ID NO: 19 - protein, 121 aa, Artificial Sequence
  h001-VH.1A
QVQLVQSGAEVKKPGASVKVSCKASGYTFNDYWMHWVRQAPGQGLEWMGYINPSSGFTKYHQNFKDRVTMTRDTSISTAYMELSRLRSDDTAVYYCARQYDYDEDWYFDVWGQGTTVTVSS

SEQ ID NO: 20 - protein, 121 aa, Artificial Sequence
  h001-VH.1B
QVQLVQSGAEVKKPGASVKVSCKASGYTFTDYWMHWVRQAPGQGLEWMGYINPSSGFTKYHQNFKDRVTMTRDTSISTAYMELSRLTSDDTAVYYCARQYDYDEDWYFDVWGQGTTVTVSS

SEQ ID NO: 21 - protein, 121 aa, Artificial Sequence
  h001-VH.1C
QVQLVQSGAEVKKPGASVKVSCKASGYTFNDYWMHWVRQAPGQGLEWMGYINPSSGFTKYHQNFKDRVTMTRDTSISTAYMELSRLTSDDTAVYYCARQYDYDEDWYFDVWGQGTTVTVSS

SEQ ID NO: 22 - protein, 121 aa, Artificial Sequence
  h001-VH.1D
QVQLVQSGAEVKKPGASVKVSCKASGYTFNDYWMHWVRQAPGQGLEWMGYINPSSGFTKYHQNFKDRVTMTADKSISTAYMELSRLTSDDTAVYYCARQYDYDEDWYFDVWGQGTTVTVSS

SEQ ID NO: 23 - protein, 121 aa, Artificial Sequence
  h001-VH.1E
QVQLVQSGAEVKKPGASVKVSCKASGYTFNDYWMHWVKQAPGQGLEWIGYINPSSGFTKYHQNFKDKATLTADKSISTAYMELSRLTSDDTAVYYCARQYDYDEDWYFDVWGQGTTVTVSS

SEQ ID NO: 24 - protein, 112 aa, Artificial Sequence
  ch-001 hVL.1 (CDR grafted)
DIQMTQSPSSLSASVGDRVTITCKSSQSLLNSRTRKNFLAWYQQKPGKAPKLLIYWASTRESGVPSRFSGSGSGTDFTLTISSLQPEDFATYYCKQSFNLFTFGQGTKLEIK

SEQ ID NO: 25 - protein, 112 aa, Artificial Sequence
  h001-VL.1A
DIQMTQSPSSLSASVGDRVTITCKSSQSLLNSRTRKNFLAWYQQKPGKAPKLLIYWASTRESGVPDRFSGSGSGTDFTLTISSLQPEDFATYYCKQSFNLFTFGQGTKLEIK

SEQ ID NO: 26 - protein, 112 aa, Artificial Sequence
  h001-VL.1B
DIQMSQSPSSLSASVGDRVTITCKSSQSLLNSRTRKNFLAWYQQKPGKAPKLLIYWASTRESGVPDRFSGSGSGTDFTLTISSLQPEDFATYYCKQSFNLFTFGQGTKLEIK

SEQ ID NO: 27 - protein, 112 aa, Artificial Sequence
  h001-VL.1C
DIVMSQSPSSLSASVGDRVTITCKSSQSLLNSRTRKNFLAWYQQKPGKSPKLLIYWASTRESGVPDRFSGSGSGTDFTLTISSLQPEDFATYYCKQSFNLFTFGQGTKLEIK

SEQ ID NO: 28 - protein, 451 aa, Artificial Sequence
  h001-4-IgG1
QVQLVQSGAEVKKPGASVKVSCKASGYTFTDYWMHWVRQAPGQGLEWMGYINPSSGFTKYHQNFKDRVTMTRDTSISTAYMELSRLRSDDTAVYYCARQYDYDEDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1413 bp, Artificial Sequence
  h001-4-IgG1
atggagtttgggctgagctggctttttcttgtcgcgattcttaagggtgtccagtgccaggtgcagctggtgcagagcggcgctgaggtgaagaagcccggagcgagcgtaaaggtgagctgcaaggccagcggatacaccttcaccgactactggatgcactgggtgaggcaggccccaggacagggcctggagtggatgggctacatcaaccccagcagcggctttaccaagtatcaccagaacttcaaagacagggtgaccatgaccagggacaccagcatcagcaccgcctacatggagctgagcaggctgaggagcgacgacaccgccgtgtactactgcgccaggcaatacgactacgacgaggactggtacttcgacgtgtggggccaaggaaccaccgtgactgtgagcagcgcttcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 30 - protein, 219 aa, Artificial Sequence
  h001-4-kappa
DIVMSQSPSSLSASVGDRVTITCKSSQSLLNSRTRKNFLAWYQQKPGKSPKLLIYWASTRESGVPDRFSGSGSGTDFTLTISSLQPEDFATYYCKQSFNLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 726 bp, Artificial Sequence
  h001-4-kappa
atggacatgcgcgtgcccgcccagctgctgggcctgctgctgctgtggttccccggctcgcgatgcgacatcgtgatgtctcagagcccatctagcctgagcgccagcgtgggcgacagggtaaccatcacctgcaagagcagccaaagcctgctgaacagcaggacccgcaagaacttcctggcttggtatcagcagaagcccggcaagtctcccaagttgctgatctactgggccagcaccagggagagcggcgtgcccgacaggttcagcggctccggcagcggcaccgacttcaccctgaccatctctagtctgcagcccgaggacttcgccacctactactgcaagcagagcttcaatctgttcaccttcggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 32 - protein, 451 aa, Artificial Sequence
  h001-4-IgG1-YTE heavy chain
QVQLVQSGAEVKKPGASVKVSCKASGYTFTDYWMHWVRQAPGQGLEWMGYINPSSGFTKYHQNFKDRVTMTRDTSISTAYMELSRLRSDDTAVYYCARQYDYDEDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1413 bp, Artificial Sequence
  h001-4-IgG1-YTE heavy chain
atggagtttgggctgagctggctttttcttgtcgcgattcttaagggtgtccagtgccaggtgcagctggtgcagagcggcgctgaggtgaagaagcccggagcgagcgtaaaggtgagctgcaaggccagcggatacaccttcaccgactactggatgcactgggtgaggcaggccccaggacagggcctggagtggatgggctacatcaaccccagcagcggctttaccaagtatcaccagaacttcaaagacagggtgaccatgaccagggacaccagcatcagcaccgcctacatggagctgagcaggctgaggagcgacgacaccgccgtgtactactgcgccaggcaatacgactacgacgaggactggtacttcgacgtgtggggccaaggaaccaccgtgactgtgagcagcgcttcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2020/0223941 A1
(43) Pub. Date: Jul. 16, 2020

(54) PHARMACEUTICAL COMPOSITION COMPRISING PCSK-9 ANTIBODY AND USE THEREOF

(71) Applicants: Jiangsu Hengrui Medicine Co., Ltd., Lianyungang, Jiangsu (CN); Shanghai Hengrui Pharmaceutical Co., Ltd., Minhang District, Shanghai (CN)

(72) Inventors: Chenmin TIAN, Minhang District, Shanghai (CN); Hao LI, Minhang District, Shanghai (CN); Xun LIU, Minhang District, Shanghai (CN)

(21) Appl. No.: 16/627,075

(22) PCT Filed: Jun. 29, 2018
(86) PCT No.: PCT/CN2018/093573

§ 371 Date: Dec. 27, 2019

(30) Foreign Application Priority Data
Jun. 30, 2017 (CN) 201710519829.6

(51) Int. Cl. C07K 16/40; A61K 47/26; A61K 9/19

(52) U.S. Cl. CPC C07K 16/40; A61K 2039/545; A61K 9/19; A61K 47/26

(57) ABSTRACT

Provided is a pharmaceutical composition comprising a PCSK9 antibody or an antigen-binding fragment thereof in a histidine buffer. In addition, the pharmaceutical composition may also comprise saccharides and nonionic surfactants.

This application claims priority of Chinese Patent Application No. CN201710519829.6, filed on Jun. 30, 2017. The entire content of the aforementioned application is hereby incorporated by reference.

### FIELD OF THE INVENTION

The invention belongs to the field of pharmaceutical formulation, in particular relates to a pharmaceutical composition comprising an anti-PCSK-9 antibody and an antigen-binding fragment thereof, and the use thereof as a medicament.

### BACKGROUND OF THE INVENTION

Hypercholesterolemia is a disease with abnormal metabolism of lipid, characterized by an increased level of serum cholesterol. Its main manifestation is the increased level of serum cholesterol, which causes cholesterol aggregation in blood vessels and consequently forms atherosclerosis. Abundant clinical and experimental research results have proven that the abnormal metabolism of lipid is closely correlated with the occurrence and development of coronary heart disease. Therefore, reducing the concentration of cholesterol in blood has become a major means for treating and preventing atherosclerosis.

With the rapid improvement of the standard of living, dyslipidemia has also become a major factor that endangers urban and rural residents in China. According to the statistics of 2012, about 40% of deaths per year in China were attributed to cardiovascular diseases. At present, the morbidity of dyslipidemia among adults in China is 18.6%, with an estimated 160 million people suffering from dyslipidemia. The morbidities of different types of dyslipidemia are as follows: 2.9% for hypercholesterolemia, 11.9% for hypertriglyceridemia, 7.4% for low high density lipoproteinemia, and 3.9% for marginally increased blood cholesterol level. In the “Chinese Experts Consensus on Prevention and Control of Chronic Disease” reached by the Branch Committee of Chronic Disease Prevention and Control, the Committee of Experts on Disease Prevention and Control, Ministry of Public Health, 2012, it was mentioned that there are 33 million of people suffering from hypercholesterolemia in China, however, from the perspective of local areas, the morbidity of dyslipidemia is far more serious than the above data.

At present, the medicaments clinically used for controlling lipid levels are mainly focused on statins. Lipitor®, as the most widely used and the best-selling cholesterol-lowering medicament, reduces the production of cholesterol by blocking the effect of a cholesterol-producing enzyme in liver, and increases the uptake of cholesterol from blood by the liver, thereby reducing the concentration of cholesterol in blood. However, Lipitor® has its disadvantages. Firstly, it will be understood from data that Lipitor® can reduce low density lipoprotein by 30% to 40%, however, an effectively reduced blood lipid level still cannot be achieved in many patients (the concentration of low density lipoprotein<50 mg/dL). Secondly, there is a difference in response rate to Lipitor® among patients of different races. For these reasons, patients still need a more effective medicine to reduce blood lipid.

As an autosomal dominant monogenic hereditary disease, familial hypercholesterolemia (FM) is clinically characterized by significant increases in total cholesterol (TC) and low density lipoprotein-cholesterol (LDL-c) in blood, xanthelasmata, corneal arcus and premature cardiovascular disease. Mutations in low density lipoprotein receptor (LDL receptor, LDLR) gene causes LDLR deficiency or absence. Consequently, LDL-c will not be transported to the liver for degradation, resulting in an increased level of LDL-c in blood. Currently, three genes, LDLR gene, apolipoprotein B100 gene and proprotein convertase subtilisin/kexin type 9 (PCSK9) gene, have been identified to be correlated with the occurrence of FM.

As a proprotein convertase, proprotein convertase subtilisin/kexin type 9 (PCSK9) belongs to a subfamily of protease K in the secretory Bacillus subtilis family. The encoded protein is synthesized as a soluble proenzyme, and intramolecularly processed in the endoplasmic reticulum by self-catalyzing. According to experimental results, PCSK9 can promote the degradation of LDL receptor and thus increases the content of LDL cholesterol in plasma. LDL receptor mediates the endocytosis process of LDL in liver, which is a main pathway to remove LDL from the circulating system. It has been found that PCSK9 gene mutations were identified in 12.5% of hypercholesterolemia (ADH) patients. There are various types of PCSK9 mutations. According to different impacts of mutations on the LDL-c level regulated by PCSK9, the mutations can be divided into two types, loss-of-function type and gain-of-function type. Among them, loss-of-function mutations are associated with low blood cholesterol level and prevent the occurrence of atherosclerotic heart disease. The mutation rate of PCSK9 associated with low cholesterol is higher in African populations than in other races. Gain-of-function mutations of PCSK9 increase plasma cholesterol level by increasing PCSK9 function and reducing LDLR expression, which can lead to severe hypercholesterolemia and premature coronary atherosclerotic heart disease. At present, it is found that gain-of-function mutations of PCSK9 include D374Y, S127R, F216L, N157K, R306S, and so on. In comparison with the PCSK9 wild type, the LDLR on the cell surface of D374Y mutants was decreased by 36%, and that of S127R mutant was decreased by 10%.

However, antibodies become unstable because of their large molecular weights, complicated structures, and susceptibility to degradation, polymerization, or undesirable chemical modification. In order to make antibodies that are suitable for administration, maintain their stability during storage and subsequent use, and exert better effects, research on stable formulations of antibodies is particularly important.

Several companies are currently developing anti-PCSK-9 antibodies and pharmaceutical formulations, such as those of CN103717237A, CN104364266A etc. However, for the new anti-PCSK-9 antibodies, there is still a need to develop a pharmaceutical (formulation) composition comprising an anti-PCSK-9 antibody that is more suitable for administration.

### DETAILED DESCRIPTION OF THE INVENTION

The present invention provides a pharmaceutical composition, comprising an anti-PCSK9 antibody or an antigen binding fragment thereof, and a buffer, wherein the buffer is preferably a histidine buffer or succinate buffer or phosphate buffer or citrate buffer, more preferably a histidine buffer, most preferably a histidine-hydrochloride buffer.

In an alternative embodiment, wherein the concentration of the anti-PCSK-9 antibody or the antigen-binding fragment thereof in the pharmaceutical composition is about 1 mg/ml to 150 mg/ml, preferably 30 mg/ml to 100 mg/ml, more preferably 50 mg/ml to 60 mg/ml, most preferably 50 mg/ml. Non-limiting examples of such concentrations of the anti-PCSK-9 antibody or the antigen-binding fragment thereof include 45 mg/ml, 46 mg/ml, 47 mg/ml, 48 mg/ml, 49 mg/ml, 50 mg/ml, 51 mg/ml, 52 mg/ml, 53 mg/ml, 54 mg/ml, 55 mg/ml, 56 mg/ml, 57 mg/ml, 58 mg/ml, 59 mg/ml, or 60 mg/ml.

In an alternative embodiment, the concentration of the buffer is about 5 mM to 50 mM, preferably 5 mM to 30 mM, non-limiting examples of concentrations of the buffer include 10 mM, 12 mM, 14 mM, 16 mM, 18 mM, 20 mM, 22 mM, 24 mM, 26 mM, 28 mM and 30 mM, more preferably 10 mM to 15 mM, most preferably 10 mM.

In an alternative embodiment, the pH of the buffer in the pharmaceutical composition is about 5.5 to 6.5, preferably about 6.0 to 6.5, non-limiting examples of pH of the buffer include about 6.0, about 6.1, about 6.2, about 6.3, about 6.4, or about 6.5.

Further, in an alternative embodiment, the pharmaceutical composition further comprises a saccharide. The “saccharide” herein comprises the conventional composition (CH₂O)n and derivatives thereof, including monosaccharides, disaccharides, trisaccharides, polysaccharides, sugar alcohols, reducing sugars, non-reducing sugars, etc. Examples of saccharides herein include glucose, sucrose, trehalose, lactose, fructose, maltose, dextran, glycerin, erythritol, glycerol, arabitol, sylitol, sorbitol, mannitol, melibiose, melezitose, raffinose, mannotriose, stachyose, maltose, lactulose, maltulose, sorbitol, maltitol, lactitol, iso-maltulose, etc. The preferred saccharide herein is a non-reducing disaccharide, more preferably trehalose or sucrose.

In an alternative embodiment, wherein the concentration of the saccharide in the pharmaceutical is about 10 mg/ml to 75 mg/ml, preferably 20 mg/ml to 60 mg/ml, more preferably about 20 mg/ml to 40 mg/ml, most preferably 25 mg/ml. Non-limiting examples of the concentration of the saccharide include 20 mg/ml, 21 mg/ml, 22 mg/ml, 23 mg/ml, 23 mg/ml, 24 mg/ml, 25 mg/ml, 26 mg/ml, 27 mg/ml, 28 mg/ml, 29 mg/ml, 30 mg/ml, 31 mg/ml, 32 mg/ml, 33 mg/ml, 34 mg/ml, 35 mg/ml, 36 mg/ml, 37 mg/ml, 38 mg/ml, 39 mg/ml, or 40 mg/ml.

In an alternative embodiment, the pharmaceutical composition further comprises a surfactant. Herein, the “surfactant” could be selected from the group consisting of a polysorbate 20, polysorbate 80, poloxamer, Triton, sodium dodecyl sulfate, sodium laurel sulfonate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyltaurate, sodium methyl oleyl-taurate, polyethyl glycol, polypropyl glycol, and copolymers of ethylene and propylene glycol etc. The preferred surfactant herein is polysorbate 20 or polysorbate 80, more preferably polysorbate 80.

In an alternative embodiment, the concentration of the surfactant in the pharmaceutical composition is about 0.05 mg/ml to 1.0 mg/ml, preferably 0.1 mg/ml to 0.4 mg/ml, non-limiting examples of concentration of the surfactant include 0.1 mg/ml, 0.15 mg/ml, 0.2 mg/ml, 0.25 mg/ml, 0.3 mg/ml, 0.35 mg/ml, 0.4 mg/ml, further preferably 0.1 mg/ml to 0.3 mg/ml, more preferably 0.1 mg/ml to 0.2 mg/ml, most preferably 0.2 mg/ml.

In an alternative embodiment, the pharmaceutical composition comprises:

(a) 1-150 mg/ml anti-PCSK-9 antibody or antigen-binding fragment thereof,

(b) 5-30 mM histidine buffer, pH about 5.5-6.5, more preferably about 6.0-6.5,

(c) 10-75 mg/ml sucrose, and

(d) 0.05-0.6 mg/ml polysorbate 80.

In an alternative embodiment, the pharmaceutical composition comprises:

(a) 50 mg/ml anti-PCSK-9 antibody or antigen-binding fragment thereof;

(b) 5-20 mM histidine buffer;

(c) 25 mg/ml sucrose; and

(d) 0.1-0.3 mg/ml polysorbate 80.

In an alternative embodiment, the pharmaceutical composition comprises:

(a) 1-150 mg/ml anti-PCSK-9 antibody or antigen-binding fragment thereof, and

(b) 5-30 mM histidine buffer; and the pH of the pharmaceutical composition is about 6.0-6.5.

In an alternative embodiment, the pharmaceutical composition comprises:

(a) 1-150 mg/ml anti-PCSK-9 antibody or antigen-binding fragment thereof;

(b) 5-30 mM histidine buffer;

(c) 10-75 mg/ml sucrose; and

(d) 0.05-0.6 mg/ml polysorbate 80, and the pH of the pharmaceutical composition is about 6.0-6.5, and the histidine buffer is preferably a histidine-hydrochloride buffer.

In an alternative embodiment, the pharmaceutical composition comprises:

(a) 50 mg/ml anti-PCSK-9 antibody or antigen-binding fragment thereof;

(b) 5-20 mM histidine buffer;

(c) 25 mg/ml of sucrose; and

(d) 0.1-0.3 mg/ml polysorbate 80, and the pH of the pharmaceutical composition is about 6.0-6.5, and the histidine buffer is preferably a histidine-hydrochloride buffer.

In an alternative embodiment, the pharmaceutical composition comprises:

(a) 50 mg/ml anti-PCSK-9 antibody or antigen-binding fragment thereof;

(b) 10 mM histidine buffer;

(c) 25 mg/ml sucrose; and

(d) 0.2 mg/ml polysorbate 80, and the pH of the pharmaceutical composition is 6.3±0.1, and the histidine buffer is preferably a histidine-hydrochloride buffer.

In an alternative embodiment, the pharmaceutical composition comprises:

(a) 150 mg/ml anti-PCSK-9 antibody or antigen-binding fragment thereof;

(b) 30 mM histidine-hydrochloride buffer;

(c) 75 mg/ml sucrose; and

(d) 0.6 mg/ml polysorbate 80; the final pH of the pharmaceutical composition is 6.3.

In an alternative embodiment, the pharmaceutical composition comprises:

(a) 50 mg/ml anti-PCSK-9 antibody or antigen-binding fragment thereof;

(b) 10 mM histidine-hydrochloride buffer, pH 6.0;

(c) 25 mg/ml sucrose; and

(d) 0.2 mg/ml polysorbate 80.

In an alternative embodiment, the pharmaceutical composition comprises:

(a) 150 mg/ml anti-PCSK-9 antibody or antigen-binding fragment thereof;

(b) 20 mM histidine-hydrochloride buffer, pH 6.5; and

(c) 70 mg/ml sucrose.

In an alternative embodiment, the pharmaceutical composition comprises:

(a) 150 mg/ml anti-PCSK-9 antibody or antigen-binding fragment thereof;

(b) 20 mM histidine-hydrochloride buffer, pH 6.5; and

(c) 70 mg/ml of α,α-dihydratetrehalose.

In an alternative embodiment, the pharmaceutical composition comprises:

(a) 50 mg/ml anti-PCSK-9 antibody or antigen-binding fragment thereof;

(b) 20 mM histidine-hydrochloride buffer, pH 6.0;

(c) 25 mg/ml sucrose; and

(d) 0.2 mg/ml polysorbate 80.

In an alternative embodiment, the pharmaceutical composition comprises:

(a) 50 mg/ml anti-PCSK-9 antibody or antigen-binding fragment thereof;

(b) 20 mM histidine-hydrochloride buffer, pH 6.5;

(c) 25 mg/ml sucrose; and

(d) 0.2 mg/ml polysorbate 80.

In an alternative embodiment, the anti-PCSK-9 antibody or the antigen binding fragment thereof in the pharmaceutical composition comprises HCDR1, HCDR2 and HCR3 having the sequences of SEQ ID NO: 12, SEQ ID NO: 13 and SEQ ID NO: 14, respectively, and

LCDR1, LCDR2 and LCDR3 having the sequences of SEQ ID NO: 15, SEQ ID NO: 16, and SEQ ID NO: 17, respectively.

In an alternative embodiment, the anti-PCSK-9 antibody or the antigen binding fragment thereof in the pharmaceutical composition is selected from the group consisting of a murine antibody, a chimeric antibody, and a humanized antibody, preferably a humanized antibody.

In an alternative embodiment, the light chain of the anti-PCSK-9 antibody in the pharmaceutical composition has at least 85%, 86%, 87%, 88%, 89%, 90%, 91%, 92%, 93%, 94%, 95%, 96%, 97%, 98%, 99% or 100% sequence identity to the amino acid sequence of the light chain of h001-4-YTE antibody, wherein the heavy chain of the anti-PCSK-9 antibody in the pharmaceutical composition has at least 85%, 86%, 87%, 88%, 89%, 90%, 91%, 92%, 93%, 94%, 95%, 96%, 97%, 98%, 99% or 100% sequence identity to the amino acid sequence of the heavy chain of h001-4-YTE antibody. The light chain sequence of h001-4-YTE antibody has the amino acid sequence of SEQ ID NO: 30, and the heavy chain sequence of h001-4-YTE antibody has the amino acid sequence of SEQ ID NO: 32.

The present invention further provides a method for preparing the pharmaceutical composition described above, comprising the step of replacing the stock solution of an anti-PCSK-9 antibody or an antigen-binding fragment thereof with a buffer. Preferably, the buffer is a histidine buffer, more preferably a histidine hydrochloride buffer. The concentration of the buffer is preferably about 5 mM to 30 mM, and non-limiting examples of concentration of the buffer include 5 mM, 6 mM, 7 mM, 8 mM, 9 mM, 10 mM, 12 mM, 14 mM, 16 mM, 18 mM, 20 mM, 22 mM, 24 mM, 26 mM, 28 mM, and 30 mM, more preferably 10 mM to 15 mM; wherein the pH of the buffer is about 6.0 to 6.5, non-limiting examples include 6.0, 6.1, 6.2, 6.3, 6.4, and 6.5.

Further, the method for preparing the pharmaceutical composition described above further comprises the step of adding sucrose and polysorbate 80 to the solution obtained from the step of replacing and then making up to final volume with buffer, wherein the concentration of the buffer is preferably about 10 mM to 20 mM, non-limiting examples of concentration of the buffer include 10 mM, 12 mM, 14 mM, 16 mM, 18 mM, and 20 mM; wherein the pH of the buffer is about 6.0 to 6.5, non-limiting examples of the pH of the buffer include 6.0, 6.1, 6.2, 6.3, 6.4, and 6.5.

The present invention further provides a method for preparing a lyophilized formulation comprising an anti-PCSK-9 antibody, which comprises the step of lyophilizing the pharmaceutical composition described above.

In an alternative embodiment, the method for preparing a lyophilized formulation containing an anti-PCSK-9 antibody sequentially comprises the steps of pre-freezing, primary drying, and secondary drying, wherein the pre-freezing is freezing from 5° C. to −40° C.-−50° C., most preferably −45° C., with no requirement for the condition of vacuum. The primary drying temperature is −14° C. to 0° C., most preferably −5° C.; the parameter of vacuum is 0.1 mBar to 0.5 mBar, most preferably 0.3 mBar. The secondary drying temperature is 20° C. to 30° C., preferably 25° C., the degree of vacuum is 0.1 mBar to 0.5 mBar, most preferably 0.3 mBar, and the degree of vacuum is reduced to 0.005 mBar-0.02 mBar, and most preferably 0.01 mBar.

The present invention further provides a lyophilized formulation comprising an anti-PCSK-9 antibody prepared by the method for preparing a lyophilized formulation containing an anti-PCSK-9 antibody described above.

In some embodiments, the lyophilized formulation is stable at 2-8° C. for at least 3 months, at least 6 months, at least 12 months, at least 18 months, or at least 24 months. In some embodiments, the lyophilized formulation is stable at 40° C. for at least 7 days, at least 14 days or at least 28 days.

The present invention further provides a method for preparing a reconstituted solution of a lyophilized formulation comprising an anti-PCSK-9 antibody, comprising the step of reconstituting the lyophilized formulation described above, wherein the solvent for reconstitution is selected from, but not limited to water for injection, physiological saline or a solution of glucose.

The present invention further provides a reconstituted solution of a lyophilized formulation comprising an anti-PCSK-9 antibody prepared by the method for preparing a reconstituted solution of a lyophilized formulation comprising an anti-PCSK-9 antibody described above.

In an alternative embodiment, the reconstituted solution containing the anti-PCSK-9 antibody of the present invention comprises a component having a concentration of 2-5 times the concentration of the component contained in the pharmaceutical composition before lyophilization, preferably 3 times.

In an alternative embodiment, the reconstituted solution comprises the anti-PCSK-9 antibody of the present invention, wherein the concentration of the anti-PCSK9 antibody or the antigen binding fragment is about 120 mg/ml to 200 mg/ml, most preferably 150 mg/ml.

In an alternative embodiment, for the reconstituted solution comprising the anti-PCSK-9 antibody of the present invention, the pH of the pharmaceutical composition is about 6.0-6.5, preferably 6.4. The pH of the reconstituted solution is related to the pH of the buffer used in the formulation of the drug substance. When the pH of the drug substance is 6.0, the pH of the final reconstituted solution is 6.3±1.

In an alternative embodiment, for the reconstituted solution comprising the anti-PCSK-9 antibody of the present invention, the concentration of the buffer is about 15 mM to 45 mM, preferably 30 mM.

In an alternative embodiment, the reconstituted solution comprising the anti-PCSK-9 antibody of the present invention further comprises disaccharide, wherein the disaccharide is preferably selected from the group consisting of trehalose and sucrose, most preferably sucrose.

In an alternative embodiment, for the reconstituted solution comprising the anti-PCSK-9 antibody of the present invention, the concentration of the disaccharide is about 55 mg/ml to 95 mg/ml, preferably about 75 mg/ml.

In an alternative embodiment, the reconstituted solution comprising the anti-PCSK-9 antibody of the present invention further comprises a surfactant, wherein the surfactant is preferably polysorbate, more preferably polysorbate 80.

In an alternative embodiment, for the reconstituted solution comprising the anti-PCSK-9 antibody of the present invention, the concentration of the surfactant is about 0.4 mg/ml to 0.8 mg/ml, preferably 0.6 mg/ml.

The invention further provides a product or kit, comprising a container containing any of the stable pharmaceutical compositions described herein. In some embodiments, it is a glass vial.

The invention further provides a use of the pharmaceutical composition, or the lyophilized formulation, or the reconstituted solution of the lyophilized formulation described above, in the formulation of a medicament for treating a PCSK9-mediated disease or disorder, wherein the disease or disorder is preferably a cholesterol-related disease; more preferably is selected from the group consisting of hypercholesterolemia, heart disease, metabolic syndrome, diabetes, coronary heart disease, apoplexy, cardiovascular disease, Alzheimer's disease and general Dyslipidemia; most preferably hypercholesterolemia, dyslipidemia, atherosclerosis, CVD or coronary heart disease.

The invention further provides a method for treating and preventing PCSK-9-related disease or disorder comprising administering to a patient in need thereof a therapeutically effective amount of the pharmaceutical composition or the lyophilized formulation or the reconstituted solution of the lyophilized formulation described above, wherein the disease or disorder is preferably a cholesterol-related disease; more preferably is selected from the group consisting of hypercholesterolemia, heart disease, metabolic syndrome, diabetes, coronary heart disease, apoplexy, cardiovascular disease, Alzheimer's disease and general Dyslipidemia; most preferably hypercholesterolemia, dyslipidemia, atherosclerosis, CVD or coronary heart disease.

The invention further provides a product, comprising a container containing the pharmaceutical composition or the lyophilized formulation or the reconstituted solution of the lyophilized formulation described above.

It should be understood that one, some, or all of the properties of the various embodiments described herein may be combined to form other embodiments of the present invention. These and other aspects of the invention will become apparent to a person skilled in the art. These and other embodiments of the invention are further described by the detailed description that follows.

### DETAILED DESCRIPTION OF THE INVENTION

In order that the present disclosure may be more readily understood, certain technical and scientific terms are specifically defined below. Unless specifically defined elsewhere in this document, all other technical and scientific terms used herein shall be taken to have the same meaning as commonly understood by one of ordinary skill in the art to which this invention belongs.

As used herein, “buffer” refers to a buffered solution that resists changes in pH by the action of its acid-base conjugate components. Examples of buffers that will control the pH in suitable range include acetate buffer, succinate buffer, gluconate buffer, histidine buffer, oxalate buffer, lactate buffer, phosphate buffer, citrate buffer, tartrate buffer, fumarate buffer, glycylglycine buffer and other organic acid buffers.

A “histidine buffer” is a buffer comprising histidine ions. Examples of the histidine buffers include histidine-hydrochloride buffer, histidine-acetate buffer, histidine-phosphate buffer and histidine-sulfate buffer, etc., preferably histidine-hydrochloride buffer. The histidine-hydrochloride buffer is prepared by histidine and hydrochloric acid, or histidine and histidine hydrochloride.

“Citrate buffer” is a buffer that includes citrate ions. Examples of the citrate buffer include citric acid-sodium citrate buffer, citric acid-potassium citrate buffer, citric acid-calcium citrate buffer and citric acid-magnesium citrate buffer, etc. A preferred citrate buffer is citric acid-sodium citrate buffer.

“Succinate buffer” is a buffer that includes succinate ions. Examples of the succinate buffer include succinic acid-sodium succinate buffer, succinic acid-potassium succinate buffer, succinic acid-calcium succinate buffer, etc. A preferred succinate buffer is succinic acid-succinate sodium buffer.

“Phosphate buffer” is a buffer that includes phosphate ions. Examples of the phosphate buffer solution include disodium hydrogen phosphate-sodium dihydrogen phosphate buffer and disodium hydrogen phosphate-potassium dihydrogen phosphate buffer, etc. A preferred phosphate buffer is disodium hydrogen phosphate-sodium dihydrogen phosphate buffer.

“Acetate buffer” is a buffer that includes acetate ions. Examples of the acetate buffer include acetic acid-sodium acetate buffer, acetate histidine buffer, acetic acid-potassium acetate buffer, acetic acid-calcium acetate buffer and acetic acid-magnesium acetate buffer, etc. A preferred acetate buffer is acetic acid-sodium acetate buffer.

“Pharmaceutical composition” refers to one containing a mixture of one or more compounds according to the present invention or a physiologically/pharmaceutically acceptable salt or produg thereof with other chemical components such as physiologically/pharmaceutically acceptable carriers and excipients. The pharmaceutical composition aims at maintaining the stability of the antibody active ingredient and promoting the administration to an organism, facilitating the absorption of the active ingredient and thereby exerting a biological effect. As used herein, “pharmaceutical composition” and “formulation” are not mutually exclusive.

“Lyophilized formulation” refers a formulation or pharmaceutical composition obtained by a vacuum freeze-drying step of a pharmaceutical composition in liquid or solution form or a liquid or solution formulation.

The lyophilization of the present invention includes pre-freezing, primary drying, and secondary drying. The purpose of pre-freezing is to freeze the product to obtain a crystalline solid. The pre-freezing temperature and the pre-freezing speed are two important process parameters. In the present invention, the pre-freezing temperature is set to −45° C., and the pre-freezing speed is set to 1° C./min. The primary drying is also known as sublimation, which is the main stage of lyophilization. The purpose of which is to maintain the shape of the product while removing the ice from the product, and minimizing damage to the product. If the temperature and vacuum degree of first drying are not appropriate, it will cause collapse of the product. Higher temperature and vacuum degree will accelerate the efficiency of lyophilization, but at the same time increase the risk of product collapsing. The temperature of the primary drying of the present invention may be a conventional temperature in the art, for example, −27° C. to 0° C., preferably −14° C. to −5° C. Secondary drying is also known as desorption, which is the main step to remove bound water from the product by pumping extreme vacuum (0.01 mbar) and increasing the temperature (20-40° C.). Since most biological products are sensitive to temperature, the temperature of secondary drying is set as the low point of the temperature range, i.e. 25° C. The lyophilization time is related to freezer, dose of lyophilized formulation, and container of lyophilized formulation. Such timing adjustment of lyophilization is well known to those skilled in the art.

The pharmaceutical composition of the present invention is capable of achieving a stable effect: the antibody which can substantially retain its physical stability and/or chemical stability and/or biological activity after storage, preferably, the pharmaceutical composition substantially retains its physical stability, chemical stability and biological activity after storage. The storage time is generally selected based on the predetermined shelf life of the pharmaceutical composition. Currently, there are a number of analytical techniques for measuring protein stability that measure the stability after storage for a selected period of time at a selected temperature.

A “stable” pharmaceutical antibody formulation is a pharmaceutical antibody formulation without any observed significant changes at a refrigerated temperature (2-8° C.) for at least 3 months, preferably 6 months, and more preferably 1 year, and even more preferably up to 2 years. Additionally, a “stable” liquid formulation includes one that exhibits desired features after storage for periods including 1 month, 3 months or 6 months at temperatures including at 25° C. and 40° C. Typical acceptable criteria for stability are as follows: typically, no more than about 10%, preferably about 5%, of the antibody monomer is degraded as measured by SEC-HPLC. The pharmaceutical antibody formulation is colorless, or clear to slightly opalescent by visual analysis. The concentration, pH and osmolality of the formulation have no more than +/−10% change. Typically, no more than about 10%, preferably about 5% of clipping is observed. Typically, no more than about 10%, preferably about 5% of aggregation is formed.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering (DLS). The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE, etc.), oxidation (evaluated by methods such as peptide mapping in conjunction with mass spectroscopy or MALDI/TOF/MS, etc.), deamidation (evaluated by methods such as ion-exchange chromatography, capillary isoelectric focusing, peptide mapping, isoaspartic acid measurement, etc.), and isomerization (evaluated by measuring the content of isoaspartic acid, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding assay.

As used herein, the single-letter code and the three-letter code for amino acids are as described in J. Biol. Chem, 243, (1968) p 3558.

As used herein, “Antibody” refers to immunoglobulin, a four-peptide chain structure connected together by disulfide bonds between two identical heavy chains and two identical light chains.

In the present invention, the antibody light chain mentioned herein further comprises a light chain constant region, which comprises a human or murine κ, λ chain or a variant thereof.

In the present invention, the antibody heavy chain mentioned herein further comprises a heavy chain constant region, which comprises human or murine IgG1, 2, 3, 4 or a variant thereof.

The variable region (Fv region), comprises about 110 of amino acids close to the N-terminus of the antibody heavy and light chain. The constant region (C region), comprising the remaining amino acids close to the C-terminus of the antibody, is relatively stable. The variable region comprises three hypervariable regions (HVRs) and four relatively conserved framework regions (FRs). The three hypervariable regions, which determine the specificity of the antibody, are also termed complementarity determining regions (CDRs). Each of the light chain variable region (LCVR) and the heavy chain variable region (HCVR) is composed of three CDR regions and four FR regions, arranged from amino terminus to carboxy terminus in the following order: FR1, CDR1, FR2, CDR2, FR3, CDR3, and FR4. Three light chain CDRs refer to LCDR1, LCDR2, and LCDR3; three heavy chain CDRs refer to HCDR1, HCDR2 and HCDR3. The number and location of CDR amino acid residues in LCVR and HCVR regions of the antibody or the antigen binding fragments herein comply with known Kabat numbering criteria (LCDR1-3, HCDE2-3), or comply with Kabat and Chothia numbering criteria (HCDR1).

The antibody of the present invention comprises a murine antibody, a chimeric antibody or a humanized antibody, preferably a humanized antibody.

The term “murine antibody” in the present invention refers to an anti-human PCSK9 monoclonal antibody prepared according to the knowledge and skill in the art. During the formulation, a test object is injected with a PCSK9 antigen, and then a hybridoma expressing antibody which possesses desired sequences or functional characteristics is isolated.

The term “chimeric antibody”, is an antibody formed by fusing a variable region of a murine antibody with the constant region of a human antibody, which can alleviate immune response induced by the murine antibody. To construct the chimeric antibody, a hybridoma that secretes a murine-specific monoclonal antibody is first established, then a variable region gene is cloned from the murine hybridoma cells. Subsequently, a constant region gene of the human antibody is cloned as desired. The murine variable region gene is ligated with human constant region gene to form a chimeric gene which is then inserted into a human vector, and finally the chimeric antibody molecule is expressed in the eukaryotic or prokaryotic industrial system. In a preferred embodiment of the present invention, the light chain of the anti-PCSK9 chimeric antibody further comprises the light chain constant regions of human κ, λ chain or a variant thereof. The heavy chain of the anti-PCSK9 chimeric antibody further comprises the heavy chain constant regions of human IgG1, IgG2, IgG3 or IgG4, or variants thereof. The constant region of a human antibody may be selected from the heavy chain constant region of human IgGl, IgG2, IgG3 or IgG4 or variants thereof, preferably comprising the heavy chain constant region of human IgG2 or IgG4, or IgG4 without ADCC (antibody-dependent cell-mediated cytotoxicity) toxicity after amino acid mutation.

The term “humanized antibody”, also known as CDR-grafted antibody, refers to an antibody generated by grafting murine CDR sequences into a variable region framework of a human antibody, namely, an antibody produced from different types of human antibody framework sequences. A humanized antibody overcomes the disadvantage of the strong antibody response induced by the chimeric antibody which carries a large amount of murine protein components. Such framework sequences can be obtained from public DNA database covering germline antibody gene sequences or published references. For example, germline DNA sequences of human heavy and light chain variable region genes can be found in “VBase” human germline sequence database (available on web www.mrccpe.com.ac.uk/vbase), as well as Kabat, E A, et al, 1991 Sequences of Proteins of Immunological Interest, 5th Ed. To avoid the decrease in activity along with the decrease in immunogenicity, the framework sequences in the variable region of the human antibody are subjected to minimal back mutations to maintain the activity. The humanized antibody of the present invention also comprises a humanized antibody to which CDR affinity maturation is performed by phage display.

An “anti-PCSK-9 antibody” is an antibody that specifically binds to PCSK-9, including but not limited to the h001 series of PCSK-9 humanized antibodies of PCT/CN2016/111053. Wherein, the h001 series of PCSK-9 humanized antibodies was screened by human PCSK-9 immunized mice and obtained by humanized transformation.

“Antigen-binding fragment” in the present invention refers to Fab fragment, Fab′ fragment, or F(ab′)2 fragment having antigen-binding activity, as well as scFv fragment binding to human PCSK9 and other fragments capable of binding PCSK9, which are formed by VH and VL regions of PCSK9 binding antibodies; it comprises one or more CDR regions selected from the group consisting of SEQ ID NO: 12 to SEQ ID NO: 17 of antibodies described in the present invention. Without a constant region, an Fv fragment comprises heavy chain variable region and light chain variable region, which is a minimal antibody fragment possessing all antigen-binding sites. Generally, an Fv antibody further comprises a polypeptide linker between the VH and VL domains, and is capable of forming a structure necessary for antigen binding. Also, different linkers can be used to connect the variable regions of two antibodies to form a polypeptide chain, named single chain antibody or single chain Fv (scFv). The term “binding to PCSK-9” in this invention means that it's capable of interacting with human PCSK-9. The term “antigen-binding sites” in the present invention, refers to continuous or discontinuous, three-dimensional sites on the antigen, recognized by the antibody or the antigen-binding fragment of the present invention.

Methods for producing and purifying antibodies and antigen-binding fragments are well known in the art and can be found, for example, in Antibody Experimental Technology Guide of Cold Spring Harbor, Chapters 5-8 and 15. The antibody or the antigen-binding fragments of the present invention is genetically engineered to introduce one or more human framework regions (FRs) to a non-human derived CDR. Human FR germline sequences can be obtained by comparing ImMunoGeneTics (IMGT) human antibody variable region germline gene database with MOE software from IMGT via their website, or from The Immunoglobulin FactsBook, 2001ISBN012441351.

The engineered antibody or antigen-binding fragments of the present invention may be prepared and purified using conventional methods. For example, cDNA sequences encoding a heavy chain and a light chain may be cloned and recombined into a GS expression vector. A recombined immunoglobulin expression vector may be stably transfected into a CHO cell. As a more recommended method well known in the art, mammalian expression systems will result in glycosylation of antibodies, typically at the highly conserved N-terminus in the Fc region. Stable clones may be obtained through expression of an antibody specifically binding to human PCSK-9. Positive clones may be expanded in serum-free culture medium for antibody production in bioreactors. Culture medium, into which an antibody has been secreted, may be purified by conventional techniques. For example, the medium may be conveniently applied to a Protein A or G Sepharose FF column that has been equilibrated with adjusted buffer. The column is washed to remove nonspecific binding components. The bound antibody is eluted by pH gradient and then pooled, and the antibody fragments are determined by SDS-PAGE. The antibody may be filtered and concentrated using conventional techniques. Soluble aggregate and multimers may be effectively removed by conventional techniques, including size exclusion or ion exchange. The obtained product may be immediately frozen, for example at −70° C., or may be lyophilized.

“Conservative modifications” or “conservative replacement or substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. It is known by those skilled in this art that, in general, single amino acid substitution in non-essential regions of a polypeptide does not substantially alter biological activity of the polypeptide (see, e.g., Watson et al. (1987) Molecular Biology of the Gene, The Benjamin/Cummings Pub. Co., p. 224 (4.sup.th Ed.)). In addition, substitutions of structurally or functionally similar amino acids are less likely to disrupt biological activity.

Amino acid “identity” refers to sequence similarity between two protein sequences or between two polypeptide sequences. When a position in both of the two compared sequences is occupied by the same amino acid residue, then the molecules are the same at that position. Examples of algorithms suitable for determining percent sequence identity and percent sequence similarity are the BLAST and BLAST 2.0 algorithms, which were described in Altschul et al. (1990) J. Mol. Biol. 215: 403-410 and Altschul et al, respectively (1977) Nucleic Acids Res. 25: 3389-3402. Software for performing BLAST analyses is available at the National Center for Biotechnology Information (www.ncbi.nlm.nih.gov/).

“Administration” and “treatment,” when applying to an animal, human, experimental subject, cell, tissue, organ, or biological fluid, refer to contacting an exogenous pharmaceutical, therapeutic, diagnostic agent, or composition with the animal, human, subject, cell, tissue, organ, or biological fluid. “Administration” and “treatment” can refer to, e.g., therapeutic, pharmacokinetic, diagnostic, research, and experimental methods. Treatment of a cell encompasses contacting a reagent with the cell, as well as contacting a reagent with a fluid, where the fluid is in contact with the cells. “Administration” and “treatment” also mean in vitro and ex vivo treatments, e.g., of a cell, by a reagent, diagnostic, binding compound, or by another cell. “Treatment,” as it applies to a human, veterinary, or a research subject, refers to therapeutic treatment, prophylactic or preventative measures, or research and diagnostic applications.

“Therapy” means to administer a therapeutic agent, such as a composition comprising any of the binding compounds of the present invention, internally or externally to a patient having one or more disease symptoms for which the agent has known therapeutic activity. Typically, the therapeutic agent is administered in an amount effective to alleviate one or more disease symptoms in a subject or population to be treated, so as to induce the regression of or inhibit the progression of such symptom(s) to any clinically measurable degree. The amount of a therapeutic agent that is effective to alleviate any particular disease symptom (also referred to “therapeutically effective amount”) may vary depending on a variety of factors, such as disease state, age, weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. Although embodiments of the present invention (e.g., therapeutic methods or articles of manufacture) may not be effective in alleviating the disease symptom(s) of interest in every patient, it should alleviate the target disease symptom(s) of interest in a statistically significant number of patients as determined by any statistical test known in the art such as the Student's t-test, the chi-square test, the U-test according to Mann and Whitney, the Kruskal-Wallis test (H-test), Jonckheere-Terpstra-test and the Wilcoxon-test.

“Effective amount” encompasses an amount sufficient to ameliorate or prevent a symptom or sign of a medical condition. Effective amount also means an amount sufficient to allow or facilitate diagnosis. An effective amount for a particular patient or veterinary subject may vary depending on factors such as the condition being treated, the general health of the patient, the route and dose of administration and the severity of side effects. An effective amount can be the maximal dose or dosing protocol that avoids significant side effects or toxic effects.

The “Tm value” refers to the heat denaturation temperature of the protein, that is, the temperature at which half of the protein is unfolded, and the spatial structure of the protein is destroyed at this time, therefore the higher the Tm value, the higher the thermal stability of the protein.

“Replacement” refers to the replacement of a solvent system that dissolves antibody proteins. For example, a buffer system of stable formulation is used to replace the high salt or hypertonic solvent system containing the antibody protein by physical modes of operation. Wherein the physical modes of operation include, but are not limited to, ultrafiltration, dialysis, or reconstitution after centrifugation.

### EXAMPLES AND TESTS

Hereinafter, the present invention is further described in detail with reference to the following examples, however, these examples are for illustrative purposes only and are not intended to limit the scope of the present invention.

In the examples of the present invention, where specific conditions are not described, the experiments are generally conducted under conventional conditions, or under conditions proposed by the material or product manufacturers. Where the source of the reagents is not specifically given, the reagents are commercially available conventional reagents.

### EXAMPLES

Formulation of PCSK-9 Antigen and Antibody

### Example 1

### Formulation of PCSK9 Antigen and Test Protein

Protein Design and Expression

Uniprot Proprotein convertase subtilisin/kexin type 9 (human PCSK9, Uniprot number: Q8MBP7) was used as a template for PCSK9 of the invention to design the amino acid sequences of antigen and test protein. PCSK9 proteins were fused with different labels such as a His tag or an immune-promoting peptide such as PADRE peptide, then cloned into pTT5 vectors (Biovector, Cat#: 102762) or pTargeT vectors (promega, A1410), respectively. The PCSK9 proteins were then transiently expressed in 293 cells or stably expressed in CHO-S cells and purified. Finally, the antigen and test protein of the invention were obtained.

PCSK9 with His tag: PCSK9-His6, used as an immunogen for immunizing mice or a detection reagent, is as follows:

SEQ ID NO: 1
MGTVSSRRSWWPLPLLLLLLLLLGPAGARAQEDEDGDYEELVLALRSEEDG
LAEAPEHGTTATFHRCAKDPWRLPGTYVVVLKEETHLSQSERTARRLQAQA
ARRGYLTKILHVFHGLLPGFLVKMSGDLLELALKLPHVDYIEEDSSVFAQS
IPWNLERITPPRYRADEYQPPDGGSLVEVYLLDTSIQSDHREIEGRVMVTD
FENVPEEDGTRFHRQASKCDSHGTHLAGVVSGRDAGVAKGASMRSLRVLNC
QGKGTVSGTLIGLEFIRKSQLVQPVGPLVVLLPLAGGYSRVLNAACQRLAR
AGVVLVTAAGNFRDDACLYSPASAPEVITVGATNAQDQPVTLGTLGTNFGR
CVDLFAPGEDIIGASSDCSTCFVSQSGTSQAAAHVAGIAAMMLSAEPELTL
AELRQRLIHFSAKDVINEAWFPEDQRVLTPNLVAALPPSTHGAGWQLFCRT
VWSAHSGPTRMATAVARCAPDEELLSCSSFSRSGKRRGERMEAQGGKLVCR
AHNAFGGEGVYAIARCCLLPQANCSVHTAPPAEASMGTRVHCHQQGHVLTG
CSSHWEVEDLGTHKPPVLRPRGQPNQCVGHREASIHASCCHAPGLECKVKE
HGIPAPQEQVTVACEEGWTLTGCSALPGTSHVLGAYAVDNTCVVRSRDVST
TGSTSEGAVTAVAICCRSRHLAQASQELQHHHHHH
Note:
Underlined sequence is a signal peptide, and the
italic part is His-tag sequence (His6-tag).

PCSK9 with PADRE peptide and His-tag: namely PCSK9-PADRE-His6, which is used as an immunogen, containing PADRE peptide that can promote immunization:

SEQ ID NO: 2
MGTVSSRRSWWPLPLLLLLLLLLGPAGARAQEDEDGDYEELVLALRSEED
GLAEAPEHGTTATFHRCAKDPWRLPGTYVVVLKEETHLSQSERTARRLQA
QAARRGYLTKILHVFHGLLPGFLVKMSGDLLELALKLPHVDYIEEDSSVF
AQSIPWNLERITPPRYRADEYQPPDGGSLVEVYLLDTSIQSDHREIEGRV
MVTDFENVPEEDGTRFHRQASKCDSHGTHLAGVVSGRDAGVAKGASMRSL
RVLNCQGKGTVSGTLIGLEFIRKSQLVQPVGPLVVLLPLAGGYSRVLNAA
CQRLARAGVVLVTAAGNFRDDACLYSPASAPEVITVGATNAQDQPVTLGT
LGTNFGRCVDLFAPGEDIIGASSDCSTCFVSQSGTSQAAAHVAGIAAMML
SAEPELTLAELRQRLIHFSAKDVINEAWFPEDQRVLTPNLVAALPPSTHG
AGWQLFCRTVWSAHSGPTRMATAVARCAPDEELLSCSSFSRSGKRRGERM
EAQGGKLVCRAHNAFGGEGVYAIARCCLLPQANCSVHTAPPAEASMGTRV
HCHQQGHVLTGCSSHWEVEDLGTHKPPVLRPRGQPNQCVGHREASIHASC
CHAPGLECKVKEHGIPAPQEQVTVACEEGWTLTGCSALPGTSHVLGAYAV

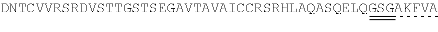

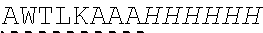

Note:
The underlined sequence is a signal peptide, the double underlined sequence is a linker, the dashed line sequence is PADRE peptide, and the italic part is His6-tag.

A fusion protein of His tag and PCSK9 with TEV cleavage site: PCSK9-TEV-His6, N-PCSK9 (N terminal PCSK9 domain), can be obtained by TEV cleavage and used as an immunogen:

SEQ ID NO: 3
MGTVSSRRSWWPLPLLLLLLLLLGPAGARAQEDEDGDYEELVLALRSEEDG
LAEAPEHGTTATFHRCAKDPWRLPGTYVVVLKEETHLSQSERTARRLQAQA
ARRGYLTKILHVFHGLLPGFLVKMSGDLLELALKLPHVDYIEEDSSVFAQS
IPWNLERITPPRYRADEYQPPDGGSLVEVYLLDTSIQSDHREIEGRVMVTD
FENVPEEDGTRFHRQASKCDSHGTHLAGVVSGRDAGVAKGASMRSLRVLNC
QGKGTVSGTLIGLEFIRKSQLVQPVGPLVVLLPLAGGYSRVLNAACQRLAR
AGVVLVTAAGNFRDDACLYSPASAPEVITVGATNAQDQPVTLGTLGTNFGR
CVDLFAPGEDIIGASSDCSTCFVSQSGTSQAAAHVAGIAAMMLSAEPELTL
AELRQRLIHFSAKDVINEAWFPEDQRVLTPNLVAALPPSTHENLYFQGAGW
QLFCRTVWSAHSGPTRMATAVARCAPDEELLSCSSFSRSGKRRGERMEAQG
GKLVCRAHNAFGGEGVYAIARCCLLPQANCSVHTAPPAEASMGTRVHCHQQ
GHVLTGCSSHWEVEDLGTHKPPVLRPRGQPNQCVGHREASIHASCCHAPGL
ECKVKEHGIPAPQEQVTVACEEGWTLTGCSALPGTSHVLGAYAVDNTCVVR
SRDVSTTGSTSEGAVTAVAICCRSRHLAQASQELQHHHHHH
Note:
The underlined sequence is a signal peptide, the
double underlined sequence is TEV cleavage site,
and the italic part is His6-tag.

PCSK9-D374Y mutant protein, with His-tag: PCSK9-D374Y-His6, which is used as a test reagent:

SEQ ID NO: 4
MGTVSSRRSWWPLPLLLLLLLLLGPAGARAQEDEDGDYEELVLALRSEEDG
LAEAPEHGTTATFHRCAKDPWRLPGTYVVVLKEETHLSQSERTARRLQAQA
ARRGYLTKILHVFHGLLPGFLVKMSGDLLELALKLPHVDYIEEDSSVFAQS
IPWNLERITPPRYRADEYQPPDGGSLVEVYLLDTSIQSDHREIEGRVMVTD
FENVPEEDGTRFHRQASKCDSHGTHLAGVVSGRDAGVAKGASMRSLRVLNC
QGKGTVSGTLIGLEFIRKSQLVQPVGPLVVLLPLAGGYSRVLNAACQRLAR
AGVVLVTAAGNFRDDACLYSPASAPEVITVGATNAQDQPVTLGTLGTNFGR
CVDLFAPGEDIIGASSYCSTCFVSQSGTSQAAAHVAGIAAMMLSAEPELTL
AELRQRLIHFSAKDVINEAWFPEDQRVLTPNLVAALPPSTHGAGWQLFCRT
VWSAHSGPTRMATAVARCAPDEELLSCSSFSRSGKRRGERMEAQGGKLVCR
AHNAFGGEGVYAIARCCLLPQANCSVHTAPPAEASMGTRVHCHQQGHVLTG
CSSHWEVEDLGTHKPPVLRPRGQPNQCVGHREASIHASCCHAPGLECKVKE
HGIPAPQEQVTVACEEGWTLTGCSALPGTSHVLGAYAVDNTCVVRSRDVST
TGSTSEGAVTAVAICCRSRHLAQASQELQHHHHHH
Note:
The underlined sequence is a signal peptide, and
the italic part is His6-tag.

PCSK9 protein inserted with biotin receiving peptide BP15 and His tag, namely PCSK9-BP15-His6. As a test reagent, BP15 peptide position can be biotinylated during expression, thus avoiding the biotin labeling in vitro and possible conformational changes.

SEQ ID NO: 5
MGTVSSRRSWWPLPLLLLLLLLLGPAGARAQEDEDGDYEELVLALRSEED
GLAEAPEHGTTATFHRCAKDPWRLPGTYVVVLKEETHLSQSERTARRLQA
QAARRGYLTKILHVFHGLLPGFLVKMSGDLLELALKLPHVDYIEEDSSVF
AQSIPWNLERITPPRYRADEYQPPDGGSLVEVYLLDTSIQSDHREIEGRV
MVTDFENVPEEDGTRFHRQASKCDSHGTHLAGVVSGRDAGVAKGASMRSL
RVLNCQGKGTVSGTLIGLEFIRKSQLVQPVGPLVVLLPLAGGYSRVLNAA
CQRLARAGVVLVTAAGNFRDDACLYSPASAPEVITVGATNAQDQPVTLGT
LGTNFGRCVDLFAPGEDIIGASSDCSTCFVSQSGTSQAAAHVAGIAAMML
SAEPELTLAELRQRLIHFSAKDVINEAWFPEDQRVLTPNLVAALPPSTHG
AGWQLFCRTVWSAHSGPTRMATAVARCAPDEELLSCSSFSRSGKRRGERM
EAQGGKLVCRAHNAFGGEGVYAIARCCLLPQANCSVHTAPPAEASMGTRV
HCHQQGHVLTGCSSHWEVEDLGTHKPPVLRPRGQPNQCVGHREASIHASC
CHAPGLECKVKEHGIPAPQEQVTVACEEGWTLTGCSALPGTSHVLGAYAV
DNTCVVRSRDVSTTGSTSEGAVTAVAICCRSRHLAQASQELQGSTSGSGL
NDIFEAQKIEWHEHHHHHH
NOTE:
The underlined sequence is a signal peptide, the double underlined sequence is the biotin receiving peptide, and the italic part is His6-tag.

PCSK9-Y is the abbreviation of PCSK9 D374Y mutant protein inserted with biotin receiving peptide BP15 and His tag, namely PCSK9-D374Y-BP15-His6, which is used as a test protein:

SEQ ID NO: 6
MGTVSSRRSWWPLPLLLLLLLLLGPAGARAQEDEDGDYEELVLALRSEED
GLAEAPEHGTTATFHRCAKDPWRLPGTYVVVLKEETHLSQSERTARRLQA
QAARRGYLTKILHVFHGLLPGFLVKMSGDLLELALKLPHVDYIEEDSSVF
AQSIPWNLERITPPRYRADEYQPPDGGSLVEVYLLDTSIQSDHREIEGRV
MVTDFENVPEEDGTRFHRQASKCDSHGTHLAGVVSGRDAGVAKGASMRSL
RVLNCQGKGTVSGTLIGLEFIRKSQLVQPVGPLVVLLPLAGGYSRVLNAA
CQRLARAGVVLVTAAGNFRDDACLYSPASAPEVITVGATNAQDQPVTLGT
LGTNFGRCVDLFAPGEDIIGASSYCSTCFVSQSGTSQAAAHVAGIAAMML
SAEPELTLAELRQRLIHFSAKDVINEAWFPEDQRVLTPNLVAALPPSTHG
AGWQLFCRTVWSAHSGPTRMATAVARCAPDEELLSCSSFSRSGKRRGERM
EAQGGKLVCRAHNAFGGEGVYAIARCCLLPQANCSVHTAPPAEASMGTRV
HCHQQGHVLTGCSSHWEVEDLGTHKPPVLRPRGQPNQCVGHREASIHASC
CHAPGLECKVKEHGIPAPQEQVTVACEEGWTLTGCSALPGTSHVLGAYAV
DNTCVVRSRDVSTTGSTSEGAVTAVAICCRSRHLAQASQELQGSTSGSGL
NDIFEAQKIEWHEHHHHHH
NOTE:
The underlined sequence is a signal peptide, the double underlined sequence is biotin receiving peptide, and the italic part is His6-tag.

LDLR extracellular domain of PCSK9 receptor protein with Flag tag and His tag, namely LDLR-ECD-Flag-His6, used as a test reagent:

SEQ ID NO: 7
MGPWGWKLRWTVALLLAAAGTAVGDRCERNEFQCQDGKCISYKWVCDGSA
ECQDGSDESQETCLSVTCKSGDFSCGGRVNRCIPQFWRCDGQVDCDNGSD
EQGCPPKTCSQDEFRCHDGKCISRQFVCDSDRDCLDGSDEASCPVLTCGP
ASFQCNSSTCIPQLWACDNDPDCEDGSDEWPQRCRGLYVFQGDSSPCSAF
EFHCLSGECIHSSWRCDGGPDCKDKSDEENCAVATCRPDEFQCSDGNCIH
GSRQCDREYDCKDMSDEVGCVNVTLCEGPNKFKCHSGECITLDKVCNMAR
DCRDWSDEPIKECGTNECLDNNGGCSHVCNDLKIGYECLCPDGFQLVAQR
RCEDIDECQDPDTCSQLCVNLEGGYKCQCEEGFQLDPHTKACKAVGSIAY
LFFTNRHEVRKMTLDRSEYTSLIPNLRNVVALDTEVASNRIYWSDLSQRM
ICSTQLDRAHGVSSYDTVISRDIQAPDGLAVDWIHSNIYWTDSVLGTVSV
ADTKGVKRKTLFRENGSKPRAIVVDPVHGFMYWTDWGTPAKIKKGGLNGV
DIYSLVTENIQWPNGITLDLLSGRLYWVDSKLHSISSIDVNGGNRKTILE
DEKRLAHPFSLAVFEDKVFWTDIINEAIFSANRLTGSDVNLLAENLLSPE
DMVLFHNLTQPRGVNWCERTTLSNGGCQYLCLPAPQINPHSPKFTCACPD
GMLLARDMRSCLTEAEAAVATQETSTVRLKVSSTAVRTQHTTTRPVPDTS
RLPGATPGLTTVEIVTMSHQALGDVAGRGNEKKPSSVRDYKDDDDKHHHH
HH
NOTE:
The underlined sequence is a signal peptide, the double underlined sequence is Flag tag, and the italic part is His6-tag.

LCDR-Fc, a fusion protein of truncated LDLR extracellular domain with hIgG1 Fc (with PCSK9 binding activity): namely LDLR-sECD-Fc (hIgG1), used as a test reagent:

SEQ ID NO: 8
MEFGLSWLFLVAILKGVQCGTNECLDNNGGCSHVCNDLKIGYECLCPDGF
QLVAQRRCEDIDECQDPDTCSQLCVNLEGGYKCQCEEGFQLDPHTKACKE
PKSSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDLIAVEWESNGQPENNYKTTPPVLDSDGSFFLYSKLTVDKS
RWQQGNVFSCSVMHEALHNHYTQKSLSLSPGK
NOTE:
The underlined sequence is a signal peptide, the double underlined sequence is truncated LDLR extracellular domain with PCSK9 binding activity (LDLR-sECD), and the italic part is hIgG1-Fc.

A fusion protein of a more truncated LDLR extracellular domain with a hIgG1 Fc (with PCSK9 binding activity): namely LDLR-ssECD-Fc (hIgG1), used as a detection reagent:

SEQ ID NO: 9
MEFGLSWLFLVAILKGVQCGTNECLDNNGGCSHVCNDLKIGYECLCPDGF
QLVAQRRCEDIDEPKSSDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
NOTE:
The underlined sequence is a signal peptide, the double underlined sequence is the more truncated LDLR extracellular domain with PCSK9 binding activity (LDLR-ssECD), and the italic part is the hIgG1-Fc.

### Example 2

### Purified Recombinant Protein of PCSK9 and LDLR Related Recombinant Protein, and Purification of Hybridoma Antibody and Recombinant Antibody

1. Purification Steps of Recombinant Proteins with His-Tag:

The samples of cell expression supernatant were centrifuged at high-speed centrifugation and impurities were removed. The buffer was replaced with PBS, and imidazole was added to a final concentration of 5 mM. A nickel column was equilibrated with 2-5 column volumes of PBS solution containing 5 mM imidazole. After buffer replacement, the supernatant sample was loaded onto the immobilized metal affinity chromatography (IMAC) column. The column was washed with PBS solution containing 5 mM imidazole, until the readout at A₂₈₀ was reduced to baseline. Then, the chromatographic column was washed with PBS+10 mM imidazole to remove nonspecific binding proteins and efflux was collected. The target protein was eluted with PBS solution containing 300 mM imidazole and the elution peak was collected. The collected elution was concentrated and further purified by gel chromatography Superdex 200 (GE) and the mobile phase was PBS. The multimer peak was removed and the elution peaks were collected. The obtained proteins were identified by electrophoresis, peptide mapping and LC-MS. PCSK9-His6 (SEQ ID NO:1), PCSK9-PADRE-His6 (SEQ ID NO: 2), PCSK9-TEV-His6 (SEQ ID NO: 3), PCSK9-D374Y-His6 (SEQ ID NO: 4), PCSK9-BP15-His6 (SEQ ID NO: 5), and PCSK9-D374Y-BP15-His6 (SEQ ID NO: 6) were obtained and used as the immunogen or test protein of the invention. PCSK9-TEV-His6 was purified and cleaved by TEV enzyme. The TEV enzyme, incompletely cleaved PCSK9-TEV-His6, or C-terminal domain fragment with His-tag was removed from the obtained product via IMAC column. The IMAC effluent was concentrated to obtain a PCSK9 segment with N-terminus domain only (abbreviated as N-PCSK9), and used as an immunogen for immunizing mice.

2. Purification Steps of Recombinant Protein of LDLR-ECD-Flag-His6 (SEQ ID NO: 7) with His Tag and Flag Tag:

Samples were centrifuged at high-speed to remove impurities, and then concentrated to a proper volume. Flag Affinity Column was equilibrated with 2-5 column volumes of 0.5× PBS buffer. After the impurities was removed, the samples of cell expression supernatant were loaded onto the column. The column was washed with 0.5× PBS, until the readout at Am was reduced to the baseline. The column was washed with PBS containing 0.3 M NaCl, and impurity proteins were eluted and collected. Target proteins were eluted with 0.1 M acetic acid (pH 3.5-4.0) and collected, and then pH value of which was adjusted to neutral. The collected elution was concentrated and further purified by gel chromatography Superdex 200 (GE) and the mobile phase was PBS. The multimer peak was removed and the elution peaks were collected. The obtained proteins were identified by electrophoresis, peptide mapping and LC-MS, and aliquoted for later use. LDLR-ECD-Flag-His6 (SEQ ID NO: 7) with FLAG/His6 tags were obtained and used for performance testing of the antibody of the present invention.

3. Purification Steps of Fusion Protein of LDLR Fc:

Samples of cell expression supernatant were centrifuged at high-speed to remove impurities, and then samples were concentrated to a proper volume and loaded onto Protein A column. The column was washed with PBS until the readout at Also was reduced to the baseline. Target proteins were eluted with 100 mM sodium acetate, pH 3.0 and then neutralized with 1 M Tris-HCl. The eluted samples were properly concentrated and were further purified by gel chromatography Superdex 200 (GE) pre-equilibrated with PBS. The peaks without multimer were collected. This method was used to purify LDLR-sECD-Fc (hIgG1) (SEQ ID NO: 8) and LDLR-ssECD-Fc (hIgG1) (SEQ ID NO: 9), both of which can be used for performance testing of anti-PCSK9 antibodies.

### Example 3

### Preparation of Anti-Human PCSK9 Hybridoma Monoclonal Antibodies

1. Immunization

The anti-human PCSK9 monoclonal antibody was produced by immunizing mice. SJL white mice, female, 6 weeks old (Beijing Vital River Laboratory Animal Technology Co., Ltd., animal permit number: SCXK (Beijing) 2012-0001) were used in this experiment and raised in a SPF laboratory. After purchase, the mice were kept in the laboratory for 1 week under 12/12 hours light/dark cycle, at a temperature of 20-25° C., and humidity 40-60%. The mice that had been adapted to the environment were immunized according to the following two schemes, with 6-10 mice per group. Immunogens were human PCSK9-His6 (SEQ ID NO: 1) with His tag, PCSK9-PADRE-His6 (SEQ ID NO: 2) and N-PCSK9 (SEQ ID NO: 3).

Scheme A: emulsifying with Freund's adjuvant (sigma Lot Num: F5881/F5506): Complete Freund's adjuvant (CFA) was used for primary immunization and Incomplete Freund's adjuvant (IFA) was used for boost immunization. The ratio of antigen to adjuvant was 1:1, 100 μg/mouse for the first immunization, and 50 μg/mouse for the booster immunization. On day 0, each mouse was intraperitoneally (IP) injected with 100 μg of emulsified antigens, once every two weeks after primary immunization, for a total of 6-8 weeks.

Scheme B: Mice were cross immunized with Titermax (sigma Lot Num: T2684) and Alum (Thremo Lot Num: 77161). The ratio of antigen to adjuvant (titermax) was 1:1, and the ratio of antigen to adjuvant (Alum) was 3:1, 10-20 μg/mouse for first immunization, and 5 μg/mouse for booster immunization. On day 0, each mouse was intraperitoneally (IP) injected with 20/10 μg emulsified antigens, once a week after primary immunization, Titermax and Alum were used alternately for a total of 6-11 weeks. Four weeks after immunization, the antigen was back or intraperitoneally injected according to the swelling conditions on the back and abdomen.

2. Cell Fusion

Mice with high antibody titer in serum (See Tests 1 and 2, ELISA method for binding PCSK9) and with the titer tending to be stationary were chosen for splenocyte fusion. 72 hours prior to fusion, the chosen mice were immunized with PCSK9-His6 via intraperitoneal injection, 10 μg/mouse. The spleen lymphocytes and myeloma cells Sp2/0 (ATCC® CRL-8287™) were fused to obtain hybridoma cells by optimized PEG-mediated fusion procedure. The fused hybridoma cells were re-suspended with HAT complete medium (RPMI-1640 medium containing 20% FBS, 1×HAT and 1×OPI), and then aliquoted into a 96-well cell culture plate (1×10⁵/150 μl/well) and incubated at 37° C. and 5% CO₂. On day 5 after fusion, HAT complete medium was added with 50 μl/well, and incubated at 37° C. and 5% CO₂. On day 7 to day 8 after fusion, based on cells growth density, the whole medium was exchanged to HT complete medium (RPMI-1640 medium containing 20% FBS, 1×HT and 1×OPI), 200 μl/well, and incubated at 37° C. and 5% CO₂.

3. Screening of Hybridoma Cells

On day 10 to day 11 after fusion, based on cell growth density, ELISA tests for PCSK9 or PCSK9-Y binding were performed (See Tests 1 and 2). Positive cells tested with ELISA were detected by blocking ELISA of PCSK9 or PCSK9-Y binding to LDLR (See Tests 3 and 4). The medium in the positive wells was exchanged and the cells were expanded to 24-well plates promptly based on cell density. Upon retest, the cell strains transferred into 24-well plate were preserved and subjected to first sub-clone. The positive cells after the first sub-clone screening (See Tests 1 and 2) were preserved and subjected to the second sub-clone. The positive cells after the second sub-clone (See Tests 1 and 2) were preserved and subjected to protein expression. Upon multiple fusions, hybridoma cells capable of blocking the binding of PCSK9 or PCSK9-Y to LDLR were obtained.

The hybridoma clone mAb-001 was obtained by screening according to blocking assay and binding assay. The antibody was further prepared by serum-free cell culturing, and the antibody was purified according to purification example for use in the test example.

The murine antibody variable region sequence of the hybridoma clone mAb-001 was as follows:

>mAb-001 VH
SEQ ID NO: 10
QVHLQQSGAELAKPGASVKLSCKASGYTFNDYWMHWVKERPGQGLEWIGYI
NPSSGFTKYHQNFKDKATLTADKSSSTAYMQLSSLTYDDSAVYYCARQYD
YDEDWYFDVWGTGTTVTVSS
>mAb-001VL
SEQ ID NO: 11
DIVMSQSPSSLAVSAGEKVTMSCKSSQSLLNSRTRKNFLAWYQQKPGQSPK
LLIYWASTRESGVPDRFTGRGSGTDFTLTISSVQAEDLAVYYCKQSFNLFT
FGSGTKLEIK
Note:
The order is FR1-CDR1-FR2-CDR2-FR3-CDR3-FR4, the
italic part is FR sequence, and the underlined is
CDR sequence.

TABLE 1
CDR region sequences of heavy chain and light
chain in Anti-PCSK-9 antibody mAb-001

| | Heavy Chain | | Light Chain | |
|---|---|---|---|---|
| mAb- | HCDR1 | DYWMH | LCDR1 | KSSQSLLNSRTRK |
| 001 | | SEQ ID NO: 12 | | NFLA SEQ ID NO: |
| | | | | 15 |
| | HCDR2 | YINPSSGFTKYHQNFKD | LCDR2 | WASTRES |
| | | SEQ ID NO: 13 | | SEQ ID NO: 16 |
| | HCDR3 | QYDYDEDWYFDV | LCDR3 | KQSFNLFT |
| | | SEQ ID NO: 14 | | SEQ ID NO: 17 |

### Example 4

### Humanization of Anti-Human PCSK9 Hybridoma Monoclonal

### Antibody

1. Selection of Humanized Framework for Hybridoma Clone mAb-001

By aligning IMGT human antibody heavy and light chain variable region germline gene database and MOE software, the heavy and light chain variable region genes with high homology with mAb-001 were selected as templates respectively. The CDRs of these two murine antibodies were respectively grafted into the corresponding human templates to form variable region sequences with the order of FR1-CDR1-FR2-CDR2-FR3-CDR3-FR4. Wherein, the amino acid residues were numbered and annotated according to the Kabat numbering system.

The humanized light chain templates of murine antibody mAb-001 are IGKV1-39*01 and hjk2.1, and the humanized heavy chain templates are IGHV1-2*02 and hjh2. The variable region sequence of humanized antibody h001-1 obtained after humanization is as follows:

>h001-1 VH
SEQ ID NO: 18
QVQLVQSGAEVKKPGASVKVSCKASGYTFTDYWMHWVRQAPGQGLEWMGYI
NPSSGFTKYHQNFKDRVTMTRDTSISTAYMELSRLRSDDTAVYYCARQYDY
DEDWYFDVWGQGTTVTVSS
>H001-1 VL
SEQ ID NO: 24
DIQMTQSPSSLSASVGDRVTITCKSSQSLLNSRTRKNFLAWYQQKPGKAPK
LLIYWASTRESGVPSRFSGSGSGTDFTLTISSLQPEDFATYYCKQSFNLFT
FGQGTKLEIK
Note:
The order of variable region is FR1-CDR1-FR2-CDR2-
FR3-CDR3-FR4, the italic is FR sequence, and the
underlined is CDR sequence.

2. Template selection and back mutation design for hybridoma clone mAb-001 are shown in Table 2. The combination of humanized sequence after back mutation of hybridoma is shown in Table 2.

TABLE 2
Template selection and back mutation design

| | | SEQ | | | SEQ |
|---|---|---|---|---|---|
| | | ID | | | ID |
| VH | | NO | VL | | NO |
| h001_VH.1 | Grafted | 18 | h001_VL.1 | Grafted | 24 |
| h001_VH.1A | T30N | 19 | h001_VL.1A | S66D | 25 |
| h001_VH.1B | R87T | 20 | h001_VL.1B | T5S, | 26 |
| | | | | S66D | |
| h001_VH.1C | T30N, R87T | 21 | h001_VL.1C | T5S, | 27 |
| | | | | S66D, | |
| | | | | Q3V, | |
| | | | | A49S | |
| h001_VH.1D | T30N, R87T, | 22 | | | |
| | R72A, T74K | | | | |
| h001_VH.1E | T30N, R87T, | 23 | | | |
| | R72A, T74K, | | | | |
| | M48I, V68A, | | | | |
| | M70L, R38K, | | | | |
| | R67K | | | | |

Note:
For example, according to Kabat numbering system, S66D means S at position 66 was back-mutated to D.
“Grafted” represents that murine antibody CDRs were grafted into human germline FR region sequences, and the sepcific sequences of the mutant variable regions are shown in Table 3:

TABLE 3
Variable region sequences of each mutant

| SEQ | |
|---|---|
| ID NO | Sequence |
| 19 | QVQLVQSGAEVKKPGASVKVSCKASGYTFNDYWMHWVRQAPGQ |
| | GLEWMGYINPSSGFTKYHQNFKDRVTMTRDTSISTAYMELSRL |
| | RSDDTAVYYCARQYDYDEDWYFDVWGQGTTVTVSS |
| 20 | QVQLVQSGAEVKKPGASVKVSCKASGYTFTDYWMHWVRQAPGQ |
| | GLEWMGYINPSSGFTKYHQNFKDRVTMTRDTSISTAYMELSRL |
| | TSDDTAVYYCARQYDYDEDWYFDVWGQGTTVTVSS |
| 21 | QVQLVQSGAEVKKPGASVKVSCKASGYTFNDYWMHWVRQAPGQ |
| | GLEWMGYINPSSGFTKYHQNFKDRVTMTRDTSISTAYMELSRL |
| | TSDDTAVYYCARQYDYDEDWYFDVWGQGTTVTVSS |
| 22 | QVQLVQSGAEVKKPGASVKVSCKASGYTFNDYWMHWVRQAPGQ |
| | GLEWMGYINPSSGFTKYHQNFKDRVTMTADKSISTAYMELSRL |
| | TSDDTAVYYCARQYDYDEDWYFDVWGQGTTVTVSS |
| 23 | QVQLVQSGAEVKKPGASVKVSCKASGYTFNDYWMHWVKQAPGQ |
| | GLEWIGYINPSSGFTKYHQNFKDKATLTADKSISTAYMELSRL |
| | TSDDTAVYYCARQYDYDEDWYFDVWGQGTTVTVSS |
| 25 | DIQMTQSPSSLSASVGDRVTITCKSSQSLLNSRTRKNFLAWYQ |
| | QKPGKAPKLLIYWASTRESGVPDRFSGSGSGTDFTLTISSLQP |
| | EDFATYYCKQSFNLFTFGQGTKLEIK |
| 26 | DIQMSQSPSSLSASVGDRVTITCKSSQSLLNSRTRKNFLAWYQ |
| | QKPGKAPKLLIYWASTRESGVPDRFSGSGSGTDFTLTISSLQP |
| | EDFATYYCKQSFNLFTFGQGTKLEIK |
| 27 | DIVMSQSPSSLSASVGDRVTITCKSSQSLLNSRTRKNFLAWYQ |
| | QKPGKSPKLLIYWASTRESGVPDRFSGSGSGTDFTLTISSLQP |
| | EDFATYYCKQSFNLFTFGQGTKLEIK |

Note:
The underlined parts of sequences are CDR regions.

TABLE 4
Humanized sequence combination of murine antibody mAb-001

| | h001_VL.1 | h001_VL.1A | h001_VL.1B | h001_VL.1C |
|---|---|---|---|---|
| h001_VH.1 | h001-1 | h001-2 | h001-3 | h001-4 |
| h001_VH.1A | h001-5 | h001-6 | h001-7 | h001-8 |
| h001_VH.1B | h001-9 | h001-10 | h001-11 | h001-12 |
| h001_VH.1C | h001-13 | h001-14 | h001-15 | h001-16 |
| h001_VH.1D | h001-17 | h001-18 | h001-19 | h001-20 |
| h001_VH.1E | h001-21 | h001-22 | h001-23 | h001-24 |

Note:
The combination of humanized antibody variable region obtained by combining various sequences and mutated sequences thereof is shown in the above Table. For example, h001-1 indicates that the variable region of humanized antibody h001-1 consists of light chain h001_VL1 and heavy chain h001_VH.1A, and so on.

3. The above humanized sequences were combined to form an antibody, wherein the heavy chain constant region is derived from human IgG1, and the light chain constant region is derived from human kappa chain. The corresponding humanized antibody was obtained, and it was verified that the PCSK9 antibodies obtained in the present invention have relatively high binding activity with PCSK9 and PCSK9-Y. Also, the antibodies can effectively block the binding of PCSK9/PCSK9-Y to LDLR.

### Example 5

### Construction and Expression of Anti-Human PCSK9 Humanized Antibodies IgG1 and IgG1-YTE Formats

The method for constructing and expressing anti-human PCSK9 humanized antibodies is as follows:

1. Primer design: Multiple primers were designed by using the online software DNAWorks (v3.2.2, http://helixweb.nih.gov/dnaworks/) to synthesize VH/VK containing gene fragments necessary for recombination: 5′-30 bp Signal peptide+VH/VK+30 bp CH1/CL-3′. The principle of primer design is as follows: if the target gene 2 differs from the target gene 1 in 2 amino acids, another primer specific for the mutation site was designed, as shown in FIG. 1.

2. Fragment splicing: according to the Manuals for TakaRa Primer STAR GXL DNA polymerase, two-step PCR amplification was performed with the multiple primers designed above and VH/VK containing gene fragments necessary for recombination was obtained.

3. Construction of expression vector pHr (with signal peptide and constant region gene (CH1-FC/CL) fragment) and restriction enzyme digestion.

Expression vector pHr (with signal peptide and constant region gene (CH1-FC/CL) fragment) were designed and constructed by utilizing the properties of some special restriction enzymes, such as BsmBI, whose recognition sequence is different from its cleavage site, as shown in FIG. 2. BsmBI was used to linearize the vector, and the gel was cut and recovered for later use.

4. Construction of the recombinant expression vector VH-CH1-FC-pHr/VK-CL-pHr.

VH/VK containing the gene fragments necessary for recombination and the recovered expression vector pHr (with the signal peptide and the constant region gene (CH1-FC/CL) fragment) digested with BsmBI enzyme were added into the DH5 alpha competent cells at a ratio of 3:1, incubated in an ice bath at 0° C. for 30 min, and heat shocked for 90 second at 42° C. Then 5 times volume of LB medium was added, incubated at 37° C. for 45 min, plated on LB-Amp plate, and cultured at 37° C. overnight. A single clone was picked and sequenced to obtain the target clone.

The antibody of this invention can be designed and constructed according to, but is not limited to, the above method. Taking h001-4 as an example, the antibody and variants thereof were designed to obtain: 1) h001-4-WT: an IgG format of h001-4, i.e., humanized sequence combination h001-4, combining heavy chain constant region derived from human IgG1 with light chain constant region derived from human kappa chain; 2) h001-4-YTE: h001-4-IgG1-YTE format, i.e., humanized sequence combination h001-4, combining heavy chain constant region of mutant human IgG1 (YTE mutation) with light chain constant region derived from human kappa chain. Mutated human IgG1 may also be other forms of mutation.

The Sequences of Constructed and Expressed Anti-Human PCSK9 Humanized Antibodies (IgG1 and IgG1-YTE Formats Thereof) are as Follows:

H001-4 IgG1 format, its heavy chain constant region is from human IgG1 and light chain constant region is from human kappa light chain.

Amino acid sequence of heavy chain (Human IgG1)
is as follows:
SEQ ID NO: 28
QVQLVQSGAEVKKPGASVKVSCKASGYTFTDYWMHWVRQAPGQGLEWMGYI
NPSSGFTKYHQNFKDRVTMTRDTSISTAYMELSRLRSDDTAVYYCARQYDY
DEDWYFDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
DNA sequence of heavy chain is as follows:
SEQ ID NO: 29
ATGGAGTTTGGGCTGAGCTGGCTTTTTCTTGTCGCGATTCTTAAGGGTGTC
CAGTGCCAGGTGCAGCTGGTGCAGAGCGGCGCTGAGGTGAAGAAGCCCGGA
GCGAGCGTAAAGGTGAGCTGCAAGGCCAGCGGATACACCTTCACCGACTAC
TGGATGCACTGGGTGAGGCAGGCCCCAGGACAGGGCCTGGAGTGGATGGGC
TACATCAACCCCAGCAGCGGCTTTACCAAGTATCACCAGAACTTCAAAGAC
AGGGTGACCATGACCAGGGACACCAGCATCAGCACCGCCTACATGGAGCTG
AGCAGGCTGAGGAGCGACGACACCGCCGTGTACTACTGCGCCAGGCAATAC
GACTACGACGAGGACTGGTACTTCGACGTGTGGGGCCAAGGAACCACCGTG
ACTGTGAGCAGCGCTTCGACCAAGGGCCCATCGGTCTTCCCCCTGGCACCC
TCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCC
CTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTT
GAGCCCAAATCTTGTGACAAAACTCACACATGCCCACCGTGCCCAGCACCT
GAACTCCTGGGGGGACCGTCAGTCTTCCTCTTCCCCCCAAAACCCAAGGAC
ACCCTCATGATCTCCCGGACCCCTGAGGTCACATGCGTGGTGGTGGACGTG
AGCCACGAAGACCCTGAGGTCAAGTTCAACTGGTACGTGGACGGCGTGGAA
GGTGCATAATGCCAAGACAAAGCCGCGGGAGGAGCAGTACACAGCACGTAC
CGTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTG
GTCAAAGGCTTCTATCCCAGCGACATCGCCGTGGAGTGGGAGAGCAATGGG
CAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTAC
ACGCAGAAGAGCCTCTCCCTGTCTCCGGGTAAATGA
h001-4-kappa
Amino acid sequence of light chain is as follows:
SEQ ID NO: 30
DIVMSQSPSSLSASVGDRVTITCKSSQSLLNSRTRKNFLAWYQQKPGKSPK
LLIYWASTRESGVPDRFSGSGSGTDFTLTISSLQPEDFATYYCKQSFNLFT
FGQGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGEC
DNA sequence of light chain is as follows:
SEQ ID NO: 31
ATGGACATGCGCGTGCCCGCCCAGCTGCTGGGCCTGCTGCTGCTGTGGTTC
CCCGGCTCGCGATGCGACATCGTGATGTCTCAGAGCCCATCTAGCCTGAGC
GCCAGCGTGGGCGACAGGGTAACCATCACCTGCAAGAGCAGCCAAAGCCTG
CTGAACAGCAGGACCCGCAAGAACTTCCTGGCTTGGTATCAGCAGAAGCCC
GGCAAGTCTCCCAAGTTGCTGATCTACTGGGCCAGCACCAGGGAGAGCGGC
GTGCCCGACAGGTTCAGCGGCTCCGGCAGCGGCACCGACTTCACCCTGACC
ATCTCTAGTCTGCAGCCCGAGGACTTCGCCACCTACTACTGCAAGCAGAGC
TTCAATCTGTTCACCTTCGGCCAGGGCACCAAGCTGGAGATCAAGCGTACG
GTGGCTGCACCATCTGTCTTCATCTTCCCGCCATCTGATGAGCAGTTGAAA
TCTGGAACTGCCTCTGTTGTGTGCCTGCTGAATAACTTCTATCCCAGAGAG
GCCAAAGTACAGTGGAAGGTGGATAACGCCCTCCAATCGGGTAACTCCCAG
GAGAGTGTCACAGAGCAGGACAGCAAGGACAGCACCTACAGCCTCACGCCA
GCAGCACCCTGACGCTGAGCAAAGCAGACTACGAGAAACACAAAGTCTTGC
GAAGTCACCCATCAGGGCCTGAGCTCGCCCGTCACAAAGAGCTTCAACAGG
GGAGAGTGTTGA
Light chain of h001-4-IgG1-YTE is h001-4-kappa:
SEQ ID NO: 30)
Amino acid sequence of heavy chain of IgGl-YTE is
as follows:
SEQ ID NO: 32
QVQLVQSGAEVKKPGASVKVSCKASGYTFTDYWMHWVRQAPGQGLEWMGYI
NPSSGFTKYHQNFKDRVTMTRDTSISTAYMELSRLRSDDTAVYYCARQYDY
DEDWYFDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTL
YITREPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
DNA sequence of heavy chain is as follows:
SEQ ID NO: 33
ATGGAGTTTGGGCTGAGCTGGCTTTTTCTTGTCGCGATTCTTAAGGGTGTC
CAGTGCCAGGTGCAGCTGGTGCAGAGCGGCGCTGAGGTGAAGAAGCCCGGA
GCGAGCGTAAAGGTGAGCTGCAAGGCCAGCGGATACACCTTCACCGACTAC
TGGATGCACTGGGTGAGGCAGGCCCCAGGACAGGGCCTGGAGTGGATGGGC
TACATCAACCCCAGCAGCGGCTTTACCAAGTATCACCAGAACTTCAAAGAC
AGGGTGACCATGACCAGGGACACCAGCATCAGCACCGCCTACATGGAGCTG
AGCAGGCTGAGGAGCGACGACACCGCCGTGTACTACTGCGCCAGGCAATAC
GACTACGACGAGGACTGGTACTTCGACGTGTGGGGCCAAGGAACCACCGTG
ACTGTGAGCAGCGCTTCGACCAAGGGCCCATCGGTCTTCCCCCTGGCACCC
TCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACC
AGCGGCGTGCACACCTTCCCGGCTGTCCTACAGTCCTCAGGACTCTACTCC
CTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTT
GAGCCCAAATCTTGTGACAAAACTCACACATGCCCACCGTGCCCAGCACCT
GAACTCCTGGGGGGACCGTCAGTCTTCCTCTTCCCCCCAAAACCCAAGGAC
ACCCTCTACATCACCCGGGAGCCTGAGGTCACATGCGTGGTGGTGGACGTG
AGCCACGAAGACCCTGAGGTCAAGTTCAACTGGTACGTGGACGGCGTGGAG
GTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAG
GAGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCTG
CCCCCATCCCGGGATGAGCTGACCAAGAACCAGGTCAGCCTGACCTGCCTG
GTCAAAGGCTTCTATCCCAGCGACATCGCCGTGGAGTGGGAGAGCAATGGG
CAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAG
GGGAACGTCTTCTCATGCTCCGTGATGCATGAGGCTCTGCACAACCACTAC
ACGCAGAAGAGCCTCTCCCTGTCTCCGGGTAAATGA
Note:
The underlined part is DNA sequence of signal
peptide.

Exemplary Preparation Process of Antibody Pharmaceutical Composition (Formulation)

Step1: A certain amount of purified anti-PCSK-9 antibody sample (such as h001-4-YTE) was taken, and the solvent (preferably by ultrafiltration) was replaced with 6 times the volume of antibody-free buffer (such as 10 mM, pH 6.0, histidine-hydrochloride buffer) by ultrafiltration membrane until the protein was concentrated to 60 mg/mL (±2 mg/mL). A certain volume of sucrose stock solution was added and mixed until the final sucrose concentration was 25 mg/mL. A certain volume of Tween-80 stock solution was added and mixed until the final Tween-80 concentration was 0.2 mg/mL. 10 mM, pH 6.0, histidine buffer was added to finalize the protein at a concentration of 50 mg/mL (±5 mg/mL). The pH of final product is about 6.3±0.1 (other formulations to be tested or stable formulations shall be prepared by similar procedures).

After the product was filtered, samples of the in-process control was taken to determine its sterility. The drug substance was passed through a 0.22 μm PVDF filter element and the filtrate was collected.

Step 2: The loading volume was adjusted to 3.6 mL, the filtrate was filled in 6 mL vial with a partial stopper, the loading difference was detected by in-process control sampling at the beginning, the middle and the end of the filling, respectively.

Step 3: The solution with a stopper was filled into the lyophilization chamber and lyophilized. The lyophilization process includes pre-freezing, primary drying and secondary drying. After the lyophilization procedure was completed, the lyophilized powder was plugged in a vacuum.

| Parameter of | | Vacuum degree |
|---|---|---|
| lyophilization process | Temperature(° C.) | (mBar) |
| Pre-freezing | 5 | / |
| | −45 | / |
| Primary drying | −5 | 0.3 |
| Secondary drying | 25 | 0.3 |
| | 25 | 0.01 |

The time used for lyophilization can be adjusted according to the actual situation. The type of lyophilizer, the loading capacity of lyophilized pharmacy, and the container of lyophilized medicament will affect the lyophilization time. Such adjustments in time are well known to those skilled in the art.

Step 4: The capping machine was turned on, aluminum cover was added, thus conducting capping.

Step 5: Visual inspection was performed to confirm the product is free from defects such as collapse, incorrect loading capacity. The label of the vial was printed and pasted; the label of carton was printed and the carton was folded, packaged and the label of the box was pasted.

### Example 6

### Screening of Buffer System

Anti-PCSK9 antibody formulation samples with a protein concentration of 1 mg/ml were prepared with the following buffer solutions.

1) 20 mM citric acid-sodium citrate, pH 4.0

2) 20 mM citric acid-sodium citrate, pH 4.5

3) 20 mM citric acid-sodium citrate, pH 5.0

4) 20 mM citric acid-sodium citrate, pH 5.5

5) 20 mM citric acid-sodium citrate, pH 6.0

6) 20 mM sodium dihydrogen phosphate-disodium hydrogen phosphate, pH 6.0

7) 20 mM sodium dihydrogen phosphate-disodium hydrogen phosphate, pH 6.5

8) 20 mM sodium dihydrogen phosphate-disodium hydrogen phosphate, pH 7.0

9) 20 mM sodium dihydrogen phosphate-disodium hydrogen phosphate, pH 7.5

10) 20 mM Tris-HCl, pH 7.5

11) 20 mM Tris-HCl, pH 8.0

12) 20 mM Tris-HCl, pH 8.5

The thermal stability of the anti-PCSK9 antibody in each formulation sample was determined by differential scanning calorimetry (DSC). The thermal denaturation midpoint temperature (Tm) analysis of the anti-PCSK-9 antibody showed that it had better thermal stability at pH≥6.0. The results are shown in Table 5. Therefore, buffers with pH between 6.0 and 6.5, such as histidine-hydrochloride, sodium dihydrogen phosphate-disodium hydrogen phosphate, sodium succinate-succinate, and citric acid-sodium citrate were selected for subsequent studies.

TABLE 5
DSC screening results of H001-4-YTE in
different buffer system and pH value

| H001-4-YTE | sucrose | | | Tmₒₙₛₑₜ | Tm |
|---|---|---|---|---|---|
| (mg/ml) | (mg/ml) | pH | Buffer system | (° C.) | (° C.) |
| 1 | N/A | 4.0 | 20 mM citric acid- | 39.17 | 66.02 |
| | | | sodium citrate | | |
| | | 4.5 | 20 mM citric acid- | 42.34 | 68.52 |
| | | | sodium citrate | | |
| | | 5.0 | 20 mM citric acid- | 44.25 | 69.77 |
| | | | sodium citrate | | |
| | | 5.5 | 20 mM citric acid- | 49.5 | 70.19 |
| | | | sodium citrate | | |
| | | 6.0 | 20 mM citric acid- | 53.57 | 70.75 |
| | | | sodium citrate | | |
| | | 6.0 | 20 mM sodium dihydrogen | 54.79 | 72.12 |
| | | | phosphate-disodium | | |
| | | | hydrogen phosphate | | |
| | | 6.5 | 20 mM sodium dihydrogen | 52.2 | 72.54 |
| | | | phosphate-disodium | | |
| | | | hydrogen phosphate | | |
| | | 7.0 | 20 mM sodium dihydrogen | 50.84 | 73.19 |
| | | | phosphate-disodium | | |
| | | | hydrogen phosphate | | |
| | | 7.5 | 20 mM sodium dihydrogen | 50.64 | 73.98 |
| | | | phosphate-disodium | | |
| | | | hydrogen phosphate | | |
| | | 7.5 | 20 mM Tris-HCl | 47.36 | 72.54 |
| | | 8.0 | 20 mM Tris-HCl | 49.09 | 73.43 |
| | | 8.5 | 20 mM Tris-HCl | 52.47 | 74.13 |

Note:
N/A represents that the ingredient is not added.

Anti-PCSK9 antibody formulation samples with a protein concentration of 1 mg/ml were prepared with the following buffer solutions.

1) 20 mM histidine-hydrochloride, pH 6.0

2) 20 mM histidine-hydrochloride, pH 6.5

3) 20 mM sodium dihydrogen phosphate-disodium hydrogen phosphate, pH 6.0

4) 20 mM sodium dihydrogen phosphate-disodium hydrogen phosphate, pH 6.5

5) 20 mM succinic acid-sodium succinate, pH 6.0

6) 20 mM citric acid-sodium citrate, pH 6.0

7) 20 mM citric acid-sodium citrate, pH 6.5

The thermal stability of anti-PCSK9 antibody in each formulation sample was determined by differential scanning calorimetry (DSC). The thermal denaturation midpoint temperature (Tm) analysis of the anti-PCSK-9 antibody showed (see Table 6) that the anti-PCSK-9 antibody was slightly more stable in the histidine, phosphate and succinate buffer systems than that in the citrate buffer system.

TABLE 6
DSC screening results of H001-4-YTE in different buffer system and pH value

| H001-4-YTE | sucrose | | | Tm₁ | Tm |
|---|---|---|---|---|---|
| (mg/ml) | (mg/ml) | pH | Buffer system | (° C.) | (° C.) |
| 1 | N/A | 6.0 | 20 mM histidine-hydrochloride | 63.51 | 72.01 |
| | | 6.5 | 20 mM histidine-hydrochloride | 65.34 | 72.45 |
| | | 6.0 | 20 mM sodium dihydrogen | 64.74 | 72.18 |
| | | | phosphate-disodium | | |
| | | | hydrogen phosphate | | |
| | | 6.5 | 20 mM sodium dihydrogen | 65.2 | 72.61 |
| | | | phosphate-disodium | | |
| | | | hydrogen phosphate | | |
| | | 6.0 | 20 mM sodium | 64.31 | 71.87 |
| | | | succinate-succinate | | |
| | | 6.0 | 20 mM citric acid-sodium | 63.73 | 70.72 |
| | | | citrate | | |
| | | 6.5 | 20 mM citric acid-sodium | 64.46 | 71.42 |
| | | | citrate | | |

Note:
N/A represents that the ingredient is not added.

### Example 7

### Screening of Saccharide in Formulation

Anti-PCSK9 antibody formulation samples with a protein concentration of 150 mg/ml were prepared with the following buffer solutions.

1) 20 mM histidine-hydrochloride, 70 mg/ml sucrose, pH 6.5

2) 20 mM histidine-hydrochloride, 70 mg/ml α,α-dihydrate trehalose, pH 6.5

Each formulation was filtered and filled into a 2 mL vial at 0.5 mL/bottle, and the vial was sealed with a stopper. Samples were taken and subjected to 40° C. high temperature experiment, low temperature illumination experiment and freeze-thaw cycle experiment (samples were placed at −20° C. for 24 h and then taken out and placed at room temperature to completely melt and mix the sample, which is a cycle). The results showed that the sucrose and trehalose have similar effects on the stability of anti-PCSK-9 antibody. Sucrose was selected as a stabilizer for anti-PCSK-9 antibody, and the results are shown in Table 7.

TABLE 7
Accelerated experimental results of different saccharides

| | | | SEC | IEC Acid | IEC Main |
|---|---|---|---|---|---|
| No. | Time | Appearance | Monomer % | Peak % | Peak % |
| 1 | Day 0 | Clear and | 99.67 | 21.87 | 60.79 |
| | | transparent | | | |
| | 40° C. | Clear and | 96.96 | 39.09 | 27.92 |
| | Day 21 | transparent | | | |
| | Freezing and | Clear and | 99.83 | 21.17 | 61.32 |
| | thawing 5 | transparent | | | |
| | times | | | | |
| | 2-8° C. | Clear and | 98.62 | 47.25 | 33.44 |
| | Illumination | transparent | | | |
| | Day 10 | | | | |
| 2 | Day 0 | Clear and | 99.93 | 22.57 | 59.38 |
| | | transparent | | | |
| | 40° C. | Clear and | 97.19 | 38.17 | 27.73 |
| | Day 21 | transparent | | | |
| | Freezing and | Clear and | 99.73 | 21.03 | 61.42 |
| | thawing 5 | transparent | | | |
| | times | | | | |
| | 2-8° C. | Clear and | 98.02 | 50.05 | 30.87 |
| | Illumination | transparent | | | |
| | Day 10 | | | | |

Anti-PCSK9 antibody formulation samples with a protein concentration of 150 mg/ml were prepared with the following buffer solutions.

1) 20 mM histidine-hydrochloride, 0 mg/ml sucrose, pH 6.5

2) 20 mM histidine-hydrochloride, 10 mg/ml sucrose, pH 6.5

3) 20 mM histidine-hydrochloride, 40 mg/ml sucrose, pH 6.5

4) 20 mM histidine-hydrochloride, 70 mg/ml sucrose, pH 6.5

The osmotic pressure value indicates that the osmotic pressure meets the minimum subcutaneous injection requirement when the concentration of sucrose is ≥70 mg/ml. The anti-PCSK-9 antibody formulation of the invention is prepared by a process of lyophilization in low concentration and reconstitution in high concentration (Three times the volume of the drug substance was lyophilized, one volume of water for injection was reconstituted, and the present invention adopts the above ratio to perform freeze-reconstitution unless specified otherwise). When the sucrose concentration in the final prescription (reconstituted formulation) is set at 75 mg/ml, it not only satisfies the osmotic pressure requirement for injection, but also facilitates the setting of the sucrose concentration in the drug substance, and the sucrose concentration of 25 mg/ml is easy to freeze-dry.

TABLE 8
Determination Results of Sucrose concentration-
osmotic pressure of H001-4-YTE formulation

| H001-4-YTE | | Saccharide | Osmotic |
|---|---|---|---|
| (mg/ml) | Saccharide | concentration(mg/ml) | pressure(mosm) |
| 150 | N/A | 0 | 44 |
| | sucrose | 10 | 71 |
| | | 40 | 153 |
| | | 70 | 290 |

Note:
N/A represents that the ingredient is not added.

### Example 8

### Screening of Surfactants in Formulations

Anti-PCSK9 antibody formulation samples with a protein concentration of 150 mg/ml were prepared with the following buffer solutions.

1) 20 mM histidine-hydrochloride, pH 6.5

2) 20 mM histidine-hydrochloride, 0.2 mg/ml polysorbate 20, pH 6.5

3) 20 mM histidine-hydrochloride, 0.4 mg/ml polysorbate 80, pH 6.5

Each formulation sample was filtered and filled into a 2 mL vial at 0.5 mL/bottle, and the vial was sealed with a stopper. Samples were placed on a 25° C. constant temperature shaker and shaken at 300 rpm. Appearance results indicated that surfactants effectively prevent aggregation of anti-PC SK-9 antibodies. There was no significant difference between polysorbate 20 and polysorbate 80 on H001-4-YTE. Polysorbate 80 was selected as the stabilizer of anti-PC SK-9 antibody formulation.

TABLE 9
Effect of Surfactant on H001-4-YTE Aggregation
shaken at 300 rpm, 25° C.

| | | | SEC | IEC Acid | IEC Main |
|---|---|---|---|---|---|
| No. | Time | Appearance | Monomer % | Peak % | Peak % |
| 1 | Day 0 | Clear and | 99.72 | 21.98 | 60.60 |
| | | transparent | | | |
| | Day 7 | Large amount | 99.88 | 20.99 | 61.63 |
| | | of particles, | | | |
| | | turbid | | | |
| 2 | Day 0 | Clear and | 99.73 | 23.35 | 58.09 |
| | | transparent | | | |
| | Day 7 | Clear and | 99.78 | 21.71 | 59.43 |
| | | transparent | | | |
| 3 | Day 0 | Clear and | 99.76 | 23.22 | 58.33 |
| | | transparent | | | |
| | Day 7 | Clear and | 99.74 | 21.83 | 59.42 |
| | | transparent | | | |

### Example 9

### Screening and Confirmation of Buffer System in Formulation

Formulation samples containing 25 mg/ml sucrose, 0.2 mg/ml polysorbate 80 and 50 mg/ml antibody was prepared using buffer solution containing 20 mM histidine-hydrochloride or 20 mM succinic acid-sodium succinate at pH 6.0 or 6.5. Each formulation was filtered and filled into a 6 mL vial at 3.6 mL/vial for lyophilization, and the vial was sealed with a halogenated butyl rubber stopper which was used for freeze-dried sterile powder. The lyophilized product was stored at 2-8° C., 25° C. and 40° C., and then reconstituted with injection water for stability analysis. The results showed that the anti-PCSK9 antibody was very stable at 2-8° C. and 25° C. However, the appearance of reconstituted formulation on day 0 showed that the succinic acid buffer at pH 6.0 had significant opalescence compared to the His system (not shown in the table), so the histidine system was selected as a buffer system for the anti-PCSK-9 antibody.

TABLE 10
Stability of H001-4-YTE lyophilized
powder at different temperatures

| | | | IEC | CE |
|---|---|---|---|---|
| | Temperature | SEC | Neutral | Non- |
| Buffer and pH | and Time | Main peak | peak | reducing |
| Succinate | 0 point | 99.5 | 57.5 | 96.4 |
| buffer pH 6.0 | 40° C., 1 month | 97.7 | 50.5 | 96.2 |
| | 25° C., 6 months | 98.9 | 57.5 | 94.0 |
| | 2-8° C., 9 momths | 99.0 | 59.5 | 95.4 |
| Histidine | 0 point | 99.6 | 54.5 | 96.9 |
| buffer pH 6.0 | 40° C., 1 month | 98.0 | 51.1 | 96.2 |
| | 25° C., 6 months | 98.7 | 56.3 | 95.1 |
| | 2-8° C., 9 momths | 99.3 | 58.8 | 95.1 |
| Histidine | 0 point | 99.7 | 57.4 | 96.4 |
| buffer pH 6.5 | 40° C., 1 month | 97.5 | 52.7 | 96.0 |
| | 25° C., 6 months | 98.6 | 55.1 | 93.2 |
| | 2-8° C., 9 momths | 98.3 | 58.9 | 94.9 |

### Example 10

### Comprehensive Screening of Formulation Components

The anti-PCSK-9 antibody formulation samples containing 50 mg/ml anti-PCSK-9 and 25 mg/ml sucrose were prepared with the following buffers at different pH, different ionic strength and containing different concentrations of surfactants.

1) 10 mM histidine-hydrochloride, 0.1 mg/ml polysorbate 80, pH 6.5

2) 10 mM histidine-hydrochloride, 0.3 mg/ml polysorbate 80, pH 6.0

3) 10 mM histidine-hydrochloride, 0.2 mg/ml polysorbate 80, pH 5.5

4) 15 mM histidine-hydrochloride, 0.3 mg/ml polysorbate 80, pH 5.5

5) 10 mM histidine-hydrochloride, 0.2 mg/ml polysorbate 80, pH 6.0

6) 15 mM histidine-hydrochloride, 0.3 mg/ml polysorbate 80, pH 6.5

7) 15 mM histidine-hydrochloride, 0.2 mg/ml polysorbate 80, pH 6.0

8) 5 mM histidine-hydrochloride, 0.3 mg/ml polysorbate 80, pH 5.5

9) 15 mM histidine-hydrochloride, 0.1 mg/ml polysorbate 80, pH 5.5

10) 5 mM histidine-hydrochloride, 0.1 mg/ml polysorbate 80, pH 6.0

11) 5 mM histidine-hydrochloride, 0.2 mg/ml polysorbate 80, pH 6.5

Each formulation sample was filtered and filled into a 6 mL vial at 3.6 mL/bottle for lyophilization, and the vial was sealed with a halogenated butyl rubber stopper which was used for lyophilized sterile powder. The lyophilized product was stored at 40° C. for stability analysis. Data analysis based on the difference in SEC monomer showed that the formulation of anti-PCSK-9 antibody using 10 mM histidine-hydrochloride buffer with pH 6.0 containing 0.2 mg/ml polysorbide 80 was more stable under the condition of 50 mg/ml protein and 25 mg/ml sucrose. After lyophilization and reconstitution, the concentration of each component in the final formulation (150 mg/ml protein, 75 mg/ml sucrose, 30 mM histidine-hydrochloride buffer, and 0.6 mg/ml polysorbate 80) was determined as three times concentration of the drug substance before lyophilization, pH 6.3±0.1.

TABLE 11
Experiment results of H001-4-YTE at high temperature

| | | | SEC | |
|---|---|---|---|---|
| No. | Time | Appearance | Monomer % | CE |
| 1 | Day 0 | Clear and transparent | 98.65 | 95.70 |
| | Day 24 | Clear and transparent | 97.98 | 96.23 |
| 2 | Day 0 | Clear and transparent | 98.76 | 95.65 |
| | Day 24 | Clear and transparent | 98.70 | 96.26 |
| 3 | Day 0 | Clear and transparent | 98.78 | 95.75 |
| | Day 24 | Clear and transparent | 97.73 | 96.34 |
| 4 | Day 0 | Clear and transparent | 98.73 | 95.66 |
| | Day 24 | Clear and transparent | 97.64 | 96.14 |
| 5 | Day 0 | Clear and transparent | 98.74 | 95.70 |
| | Day 24 | Clear and transparent | 97.53 | 96.30 |
| 6 | Day 0 | Clear and transparent | 98.72 | 95.74 |
| | Day 24 | Clear and transparent | 97.49 | 96.10 |
| 7 | Day 0 | Clear and transparent | 98.75 | 95.77 |
| | Day 24 | Clear and transparent | 97.50 | 96.11 |
| 8 | Day 0 | Clear and transparent | 98.70 | 95.75 |
| | Day 24 | Clear and transparent | 97.32 | 95.89 |
| 9 | Day 0 | Clear and transparent | 98.37 | 95.78 |
| | Day 24 | Clear and transparent | 97.68 | 96.19 |
| 10 | Day 0 | Clear and transparent | 98.80 | 95.73 |
| | Day 24 | Clear and transparent | 97.26 | 96.08 |
| 11 | Day 0 | Clear and transparent | 98.44 | 95.67 |
| | Day 24 | Clear and transparent | 97.06 | 96.01 |

### Example 11

### Temperature Optimization of the Primary Drying

Anti-PCSK9 antibody formulation samples with an anti-PCSK9 antibody concentration of 50 mg/ml, 25 mg/ml sucrose, 0.2 mg/ml polysorbate 80 were prepared with the 10 mM histidine-hydrochloride at pH 6.0. The antibody was filled into a 6 mL vial with 3.6 mL/vial and lyophilized at a primary drying temperature of −14° C., and −5° C. respectively, and the vial was sealed with a stopper for lyophilization. The reconstituted samples before and after lyophilization were compared. The results show that −5° C. is the preferred primary drying temperature of the lyophilization process.

TABLE 12
Comparison of H001-4-YTE samples prepared with different
drying processes before and after freeze-drying

| | | Lyophilized | Lyophilized |
|---|---|---|---|
| | Before | at −14° C.,and | at −5° C., and |
| Test items | freeze-drying | Reconstituted | Reconstituted |
| Concentration | 52.3 mg/ml | 152.5 mg/ml | 148.0 mg/ml |
| pH | 6.32 | 6.36 | 6.32 |
| Appearance | Clear and | Clear and | Clear and |
| | transparent | transparent | transparent |
| SEC Monomer(%) | 99.8 | 99.8 | 99.9 |
| CE Non-reducing (%) | 96.3 | 96.2 | 96.3 |
| IEC Main Peak (%) | 61.3 | 61.4 | 60.5 |
| Moisture content | N/A | 0.5% | 0.6% |

### Example 12

### Determination of Compatibility Between Formulation and Containers of Different Materials

H001-4-YTE was prepared at 50 mg/mL in a solution comprising 10 mM histidine-hydrochloride, pH 6.0, with 25 mg/ml sucrose and 0.2 mg/ml polysorbate 80. The formulation samples were filled in glass bottles, liquid storage bags and 316 L stainless steel tanks respectively, and placed at 2-8° C. for 24 hours. Protein content and purity analysis showed that H001-4-YTE was stable within 24 hours. The formulation was compatible with the 316 L stainless steel tank, glass bottle and liquid storage bag.

TABLE 13
Stability of H001-4-YTE in different contact materials

| | | | | | | | Non- | |
|---|---|---|---|---|---|---|---|---|
| | | | | | IEC (%) | | Reducing | |
| | | | SEC (%) | | Acid | Neutral | CE-SDS | Protein |
| Material | Temperature | Time | Monomer | Polymer | Peak | peak | (%) | Content |
| Stainless | 2-8° C. | 0 | 99.46 | 0.09 | 20.77 | 61.82 | 94.70 | 52.27 |
| steel | | 8 h | 99.42 | 0.08 | 20.69 | 62.02 | 96.17 | 51.10 |
| | | 24 h | 99.75 | 0.00 | 20.64 | 62.21 | 95.03 | 52.89 |
| Liquid | 2-8° C. | 0 | 99.46 | 0.09 | 20.77 | 61.82 | 94.70 | 52.27 |
| storage | | 8 h | 99.47 | 0.14 | 20.67 | 61.94 | 96.19 | 51.15 |
| bag | | 24 h | 99.76 | 0.02 | 20.75 | 61.89 | 96.12 | 52.41 |
| Glass | 2-8° C. | 0 | 99.46 | 0.09 | 20.77 | 61.82 | 94.70 | 52.27 |
| bottle | | 8 h | 99.72 | 0.06 | 20.54 | 62.24 | 96.30 | 51.02 |
| | | 24 h | 99.62 | 0.00 | 20.71 | 61.95 | 96.39 | 51.14 |

### Example 13

### Determination of Compatibility of Formulation with Different Filters

H001-4-YTE was prepared at 50 mg/mL in a solution comprising 10 mM histidine hydrochloride buffer, pH 6.0, with 25 mg/ml sucrose and 0.2 mg/ml polysorbate 80. The formulation sample was passed through 0.22 μm PES filters and PVDF filters, and samples were taken at 30 min and 1 h for testing. Analysis of protein content, appearance and purity indicated that H001-4-YTE was stable within 1 hour of contact with the filter. The formulation is compatible with both PES and PVDF filters.

TABLE 14
Stability of H001-4-YTE in different materials of filter membranes

| | | | | | | | Non- | |
|---|---|---|---|---|---|---|---|---|
| Materials | | | | | IEC (%) | | reducing | |
| of filter | | | SEC (%) | | Acid | Neutral | CE-SDS | Protein |
| membranes | Time | Appearance | Monomer | Fragment | Peak | Peak | (%) | Content |
| PES | 0 h | Clear | 99.30 | 0.69 | 20.10 | 63.60 | 95.77 | 53.15 |
| | | and | | | | | | |
| | | transparent | | | | | | |
| | 0.5 h | Clear | 99.34 | 0.66 | 19.90 | 63.60 | 96.09 | 53.97 |
| | | and | | | | | | |
| | | transparent | | | | | | |
| | 1 h | Clear | 99.00 | 1.00 | 20.30 | 63.30 | 96.03 | 53.11 |
| | | and | | | | | | |
| | | transparent | | | | | | |
| PVDF | 0 h | Clear | 99.11 | 0.89 | 20.10 | 63.60 | 95.84 | 52.23 |
| | | and | | | | | | |
| | | transparent | | | | | | |
| | 0.5 h | Clear | 99.27 | 0.73 | 20.00 | 63.90 | 96.07 | 53.48 |
| | | and | | | | | | |
| | | transparent | | | | | | |
| | 1 h | Clear | 99.21 | 0.79 | 20.00 | 63.90 | 96.02 | 52.42 |
| | | and | | | | | | |
| | | transparent | | | | | | |

### Example 14

### Other Alternative Formulations of Formulation

The pharmaceutical composition of the present invention can be used as a drug substance or an injection solution of a pharmaceutical formulation directly. When the pharmaceutical composition of the present invention is used as a stock solution of a pharmaceutical formulation, a lyophilized formulation is prepared by a lyophilization process and reconstituted into an injection solution for clinical use. The lyophilized formulation of the invention is prepared according to a process of lyophilization in low concentration and reconstitution in high concentration, that is, the low concentration of the pharmaceutical formulation solution is lyophilized to obtain a lyophilized formulation, and the lyophilized formulation is reconstituted into a higher concentration of pharmaceutical composition for clinical use. The lyophilized formulation has a longer stability than the liquid formulation. For the drug substance of pharmaceutical formulation used for lyophilized formulation, the higher the concentration of each component is, the longer the lyophilization time required. When various indicators setting of the lyophilization process are considered comprehensively, the concentration of each component of the injection after reconstitution of the pharmaceutical formulation is usually 2-5 times of the concentration of each component of the injection after reconstitution, and 3 times in the preferred embodiment of the present invention.

The stable pharmaceutical formulation provided by the present invention comprises: an anti-PCSK-9 antibody protein (non-limiting embodiment such as h001-4-YTE) and a combination of any of the following stable buffers:

(1) Antibody h001-4-YTE 150 mg/ml, 75 mg/ml sucrose, 0.6 mg/ml polysorbate 80, and 30 mM histidine-hydrochloride buffer, pH 6.4;

(2) Antibody h001-4-YTE 150 mg/ml, 75 mg/ml sucrose, 0.6 mg/ml polysorbate 80, and 30 mM histidine-hydrochloride buffer, pH 6.2;

(3) Antibody h001-4-YTE 150 mg/ml, 75 mg/ml sucrose, 0.6 mg/ml polysorbate 80, and 30 mM histidine-hydrochloride buffer, pH 6.3;

(4) Antibody h001-4-YTE 50 mg/ml, 25 mg/ml sucrose, 0.2 mg/ml polysorbate 80, and 10 mM histidine-hydrochloride buffer, pH 6.4;

(5) Antibody h001-4-YTE 50 mg/ml, 25 mg/ml sucrose, 0.2 mg/ml polysorbate 80, and 10 mM histidine-hydrochloride buffer, pH 6.3;

(6) Antibody h001-4-YTE 50 mg/ml, 25 mg/ml sucrose, 0.2 mg/ml polysorbate 80, and 10 mM histidine-hydrochloride buffer, pH 6.2;

(7) Antibody h001-4-YTE 50 mg/ml, 25 mg/ml sucrose, 0.2 mg/ml polysorbate 80, and 10 mM histidine-hydrochloride buffer, final pH 6.1;

(8) Antibody h001-4-YTE 50 mg/ml, 25 mg/ml sucrose, 0.2 mg/ml polysorbate 80, and 10 mM histidine-hydrochloride buffer, pH 6.0;

(9) Antibody h001-4-YTE 50 mg/ml, 25 mg/ml sucrose, 0.2 mg/ml polysorbate 80, and 10 mM histidine-hydrochloride buffer, pH 6.5;

(10) Antibody h001-4 50 mg/ml, 25 mg/ml sucrose, 0.2 mg/ml polysorbate 80, and 10 mM histidine-hydrochloride buffer, pH 6.3;

(11) Antibody h001-4 50 mg/ml, 25 mg/ml sucrose, 0.2 mg/ml polysorbate 80, and 10 mM histidine-hydrochloride buffer, pH 6.2;

(12) Antibody h001-4 50 mg/ml, 25 mg/ml sucrose, 0.2 mg/ml polysorbate 80, and 10 mM histidine-hydrochloride buffer, pH 6.4;

(13) Antibody h001-4-YTE 50 mg/ml, 25 mg/ml sucrose, 0.1 mg/ml polysorbate 80, and 20 mM histidine-hydrochloride buffer, pH 6.3;

(14) Antibody h001-4-YTE 50 mg/ml, 25 mg/ml sucrose, 0.2 mg/ml polysorbate 80, and 15 mM histidine-hydrochloride buffer, pH 6.2;

(15) Antibody h001-4-YTE 50 mg/ml, 25 mg/ml sucrose, 0.2 mg/ml polysorbate 80, and 20 mM histidine-hydrochloride buffer, pH 6.4.

(16) Antibody h001-4-YTE 30 mg/ml, 10 mg/ml sucrose, 0.05 mg/ml polysorbate 80, and 5 mM histidine-hydrochloride buffer at pH 5.5;

(17) Antibody h001-4-YTE 70 mg/ml, 75 mg/ml sucrose, 0.6 mg/ml polysorbate 80, and 30 mM histidine-hydrochloride buffer at pH 6.5;

(18) Antibody h001-4-YTE 45 mg/ml, 20 mg/ml sucrose, 0.1 mg/ml polysorbate 80, and 8 mM histidine-hydrochloride buffer at pH 6.0;

(19) Antibody h001-4-YTE 55 mg/ml, 40 mg/ml sucrose, 0.3 mg/ml polysorbate 80, and 15 mM histidine-hydrochloride buffer at pH 6.2.

The pharmaceutical composition of the present invention can be prepared into a corresponding lyophilized formulation by a lyophilization process, and the lyophilized formulation can be reconstituted with an injection water to obtain the following pharmaceutical composition for clinical use:

(1) Anti-PCSK-9 antibody protein 150 mg/ml, 75 mg/ml sucrose, 0.6 mg/ml polysorbate 80, and 20 mM histidine-hydrochloride buffer, pH 6.3±0.1;

(2) Anti-PCSK-9 antibody protein 120 mg/ml, 55 mg/ml sucrose, 0.4 mg/ml polysorbate 80, and 10 mM histidine-hydrochloride buffer, pH 6.0;

(3) Anti-PCSK-9 antibody protein 200 mg/ml, 95 mg/ml sucrose, 0.8 mg/ml polysorbate 80, and 30 mM histidine-hydrochloride buffer, pH 6.5.

Although the specific embodiments of the present invention have been described above, those skilled in the art will understand that these are merely illustrative. Many changes or modifications may be made to these embodiments without departing from the principle and essence of the invention. Therefore, the scope of the invention is defined by the appended claims.

1-28. (canceled)

29. A pharmaceutical composition comprising:
an anti-PCSK-9 antibody or antigen-binding fragment thereof at a concentration of 1 mg/ml to 150 mg/ml;
a buffer at a concentration of 5 mM to 30 mM, wherein the buffer is selected from the group consisting of a histidine buffer, a succinate buffer, a phosphate buffer and a citrate buffer;
a disaccharide at a concentration of 10 mg/ml to 75 mg/ml; and
a surfactant at a concentration of 0.05 mg/ml to 0.6 mg/ml;
wherein the pharmaceutical composition has a pH of 5.5 to 6.5.

30. The pharmaceutical composition of claim 29, wherein the anti-PCSK-9 antibody or antigen binding fragment thereof comprises a heavy chain variable region comprising HCDR1, HCDR2 and HCDR3 having the amino acid sequences of SEQ ID NO: 12, SEQ ID NO: 13 and SEQ ID NO: 14, respectively, and a light chain variable region comprising LCDR1, LCDR2 and LCDR3 having the amino acid sequences of SEQ ID NO: 15, SEQ ID NO: 16, and SEQ ID NO: 17, respectively.

31. The pharmaceutical composition of claim 29, wherein the buffer is the histidine buffer.

32. The pharmaceutical composition of claim 29, wherein the disaccharide is trehalose or sucrose.

33. The pharmaceutical composition of claim 29, wherein the surfactant is a polysorbate.

34. A pharmaceutical composition, comprising:
an anti-PCSK-9 antibody or antigen binding fragment thereof at a concentration of 30 mg/ml to 100 mg/ml, wherein the anti-PCSK-9 antibody or antigen-binding fragment thereof comprises a heavy chain variable region comprising HCDR1, HCDR2 and HCDR3 having the amino acid sequences of SEQ ID NO: 12, SEQ ID NO: 13 and SEQ ID NO: 14, respectively, and a light chain variable region comprising LCDR1, LCDR2 and LCDR3 having the amino acid sequences of SEQ ID NO: 15, SEQ ID NO: 16, and SEQ ID NO: 17, respectively;
a histidine buffer at a concentration of 5 mM to 20 mM;
a disaccharide at a concentration of 20 mg/ml to 40 mg/ml, wherein the disaccharide is selected from the group consisting of trehalose and sucrose; and
a polysorbate at a concentration of 0.1 mg/ml to 0.4 mg/ml;
wherein the pharmaceutical composition has a pH of 6.0 to 6.5.

35. The pharmaceutical composition of claim 34, comprising
the anti-PCSK-9 antibody or antigen binding fragment thereof at a concentration of 50 mg/ml;
a histidine-hydrochloride buffer at a concentration of 10 mM;
sucrose at a concentration of 25 mg/ml; and
polysorbate 80 at a concentration of 0.2 mg/ml;
wherein the pharmaceutical composition has a pH of 6.0.

36. A method of preparing the pharmaceutical composition of claim 29, the method comprising:
replacing a stock solution of a composition comprising the anti-PCSK-9 antibody or antigen-binding fragment thereof with the buffer at the concentration of 5 mM to 30 mM, wherein the buffer has the pH of 5.5 to 6.5; and
adding the disaccharide and the surfactant to the composition to obtain the pharmaceutical composition.

37. A method for preparing a lyophilized formulation, the method comprising freeze-drying the pharmaceutical composition of claim 29.

38. A lyophilized formulation prepared by the method of claim 37.

39. A method for preparing a reconstituted solution, comprising reconstituting the lyophilized formulation of claim 38 with a solvent for reconstitution, wherein the solvent for reconstitution is water for injection.

40. The reconstituted solution prepared by the method of claim 39.

41. The reconstituted solution of claim 40, comprising the anti-PCSK-9 antibody or antigen-binding fragment thereof at a concentration of 120 mg/ml to 200 mg/ml, the buffer at a concentration of 15 mM to 30 mM, the disaccharide at a concentration of 55 mg/ml to 95 mg/ml, and the surfactant at a concentration of 0.4 mg/ml to 0.8 mg/ml, wherein the reconstituted solution has a pH of 6.0 to 6.5.

42. The reconstituted solution of claim 41, comprising the anti-PCSK-9 antibody or antigen-binding fragment thereof at a concentration of 150 mg/ml, the histidine buffer at a concentration of 30 mM, sucrose at a concentration of 75 mg/ml, and polysorbate 80 at a concentration of 0.6 mg/ml, wherein the reconstituted solution has a pH of 6.3.

43. A method of treating a PCSK-9-related disease or disorder comprising administering to a patient in need thereof a therapeutically effective amount of the pharmaceutical composition of claim 29, wherein the disease or disorder is selected from the group consisting of hypercholesterolemia, heart disease, metabolic syndrome, diabetes, coronary heart disease, apoplexy, cardiovascular disease, Alzheimer's disease and general Dyslipidemia.

44. A method of treating a PCSK-9-related disease or disorder comprising administering to a patient in need thereof a therapeutically effective amount of the pharmaceutical composition of claim 34, wherein the disease or disorder is selected from the group consisting of hypercholesterolemia, heart disease, metabolic syndrome, diabetes, coronary heart disease, apoplexy, cardiovascular disease, Alzheimer's disease and general Dyslipidemia.

45. A method of treating a PCSK-9-related disease or disorder comprising administering to a patient in need thereof a therapeutically effective amount of the reconstituted solution of claim 40, wherein the disease or disorder is selected from the group consisting of hypercholesterolemia, heart disease, metabolic syndrome, diabetes, coronary heart disease, apoplexy, cardiovascular disease, Alzheimer's disease and general Dyslipidemia.

46. A method of treating a PCSK-9-related disease or disorder comprising administering to a patient in need thereof a therapeutically effective amount of the reconstituted solution of claim 41, wherein the disease or disorder is selected from the group consisting of hypercholesterolemia, heart disease, metabolic syndrome, diabetes, coronary heart disease, apoplexy, cardiovascular disease, Alzheimer's disease and general Dyslipidemia.

47. A method of treating a PCSK-9-related disease or disorder comprising administering to a patient in need thereof a therapeutically effective amount of the reconstituted solution of claim 42, wherein the disease or disorder is selected from the group consisting of hypercholesterolemia, heart disease, metabolic syndrome, diabetes, coronary heart disease, apoplexy, cardiovascular disease, Alzheimer's disease and general Dyslipidemia.

48. A kit comprising a container and the pharmaceutical composition of claim 29.
